(19) United States
(12) Patent Application Publication
(10) Pub. No.: US 2008/0299545 A1
(43) Pub. Date: Dec. 4, 2008

(54) CHROMATOGRAPHIC METHODS FOR ASSESSING ADENOVIRUS PURITY

(76) Inventors: Shuyuan Zhang, Boyds, MD (US); Peter Clarke, Lincroft, NJ (US)

Correspondence Address: FULBRIGHT & JAWORSKI L.L.P., 600 CONGRESS AVE., SUITE 2400, AUSTIN, TX, 78701, US

(21) Appl. No.: 12/043,557

(22) Filed: Mar. 6, 2008

Related U.S. Application Data
(60) Provisional application No. 60/893,340, filed on Mar. 6, 2007.

(51) Int. Cl. C12Q 1/70

(52) U.S. Cl. 435/5

(57) ABSTRACT

Methods for determining the quantity, quality and purity of a previously purified virus sample are disclosed. Such methods, which include the use of high performance size exclusion chromatography to determine these attributes are also disclosed.

This application claims priority to U.S. Provisional Patent application Ser. No. 60/893,340 filed Mar. 6, 2007, which is incorporated herein by reference in its entirety.

### BACKGROUND OF THE INVENTION

I. Field of the Invention

The present invention relates generally to the field of protein and virus purification. More particularly, it concerns methods for assaying proteins and/or virus particles to determine their level of purity following a purification process.

II. Description of the Related Art

A variety of cancer and genetic diseases currently are being addressed by protein or gene therapy. The basis of such therapy may include delivery of a purified protein or viral vector encoding a therapeutic or diagnostic gene. Viruses are highly efficient at nucleic acid delivery to specific cell types, while often avoiding detection by the infected host's immune system. These features make certain viruses attractive candidates as gene-delivery vehicles for use in gene therapies (Robbins and Ghivizzani, 1998; Cristiano et al., 1998).

Certain viruses, such as modified adenoviruses that are replication incompetent and therefore non-pathogenic are being used as vehicles to deliver therapeutic genes for a number of metabolic and oncologic disorders. These adenoviral vectors may be particularly suitable for disorders such as cancer that would best be treated by transient therapeutic gene expression since the DNA is not integrated into the host genome and the transgene expression is limited. Adenoviral vector may also be of significant benefit in gene replacement therapies, wherein a genetic or metabolic defect or deficiency is remedied by providing for expression of a replacement gene encoding a product that remedies the defect or deficiency.

Traditionally, adenoviruses are produced in commercially available tissue culture flasks or “cellfactories.” Adenoviral vector production has generally been performed in culture devices that supply culture surfaces for attachment of the helper 293 cells, such as T-flasks. Virus infected cells are harvested and freeze-thawed to release the viruses from the cells in the form of crude cell lysate. The produced crude cell lysate (CCL) is then purified by double CsCl gradient ultracentrifugation. The typically reported virus yield from 100 single tray cell factories is about 6×10¹² PFU.

For many applications involving adenoviral vectors, such as research or clinical applications, it may be desirable to both purify the adenovirus preparation and assay the level of purity. Several methods are known to exist for the purification of such vectors. One current method for the purification of adenovirus is density gradient centrifugation. While useful in research settings, this method has received less attention for larger scale clinical applications due to its inherent limitations. However, more recent studies have shown that column chromatography may be used in the purification of recombinant adenovirus More specifically, Huyghe et al., 1996, reported adenoviral vector purification using ion exchange chromatography in conjunction with metal chelate affinity chromatography. Virus purity similar to that from CsCl gradient ultracentrifugation was reported. Unfortunately, only 23% of virus was recovered after the double column purification process. Process factors that contribute to this low virus recovery are the freeze/thaw step utilized by the authors to lyse cells in order to release the virus from the cells and the two column purification procedure.

Column chromatography techniques may employ any number of different columns to achieve the desired level of purification. For example, ion-exchange, affinity chromatography and size exclusion chromatography are becoming useful tools in the art of protein and virus purification. Additionally chromatography techniques have been demonstrated for large-scale purification of adenoviral vectors such as international patent applications: WO 96/27677, WO 97/08298, WO 98/00524 and WO 98/22588. Application WO 98/00524, for example, discloses a method of purification using anion-exchange resin Source 15Q which makes it possible to obtain, in a single chromatographic step, adenovirus preparations whose purity is at least equivalent to that obtained from preparations purified by cesium chloride gradient ultracentrifugation.

Additionally, the use of size exclusion chromatography for purification of various plant viruses has been demonstrated either as a stand alone technique or to augment density gradient centrifugation (Albrechtsen et al., 1990; and Hewish et al., 1983). Size exclusion chromatography has also been demonstrated for animal viruses (Crooks et al., 1990).

U.S. Pat. No. 6,261,823 discloses a method of purifying adenovirus which comprises subjecting a virus preparation to anion exchange chromatography followed by size exclusion chromatography. U.S. Pat. No. 6,194,191 discloses methods of purifying adenovirus using low perfusion rates during cell culture, a detergent lysis step, and/or a single chromatography step.

In fact, some techniques involve a combination of approaches. For example, U.S. Pat. No. 5,837,520 discloses a method of purifying adenovirus which comprises treating the cell lysate with a nuclease, followed by (1) anion exchange and (2) metal ion chromatography.

Despite the availability of the above-discussed techniques, adenovirus preparations produced by these methods still contain impurities introduced as a result of the batch process for the production of the initial adenovirus preparation. For example, process contaminants such as bovine serum albumin (BSA), host cell proteins, viral contaminants, and nucleic acids are all present in the crude adenovirus preparation and many of these process impurities may remain in the adenovirus preparation even after the preparation has been subjected to various chromatographic purifications.

In addition, even when purification is thought to be satisfactory, methods are needed to assay the adenovirus product to ensure purity and quantify the product for use in research or clinical applications. Previously, adenovirus product purity has been measured by ion exchange HPLC. However, because the adenovirus product is purified using the same type ion exchange chromatography, the use of the same type of column is may not be able to detect residual impurities in the adenovirus product and provide additional purity information. Clearly, there is a demand for improved methods of assaying the purity of a given virus sample, particularly a sample designated for clinical applications. For example, improved purification assays may include the exploitation of the difference in molecular weight of a virus and smaller impurities to analyze the purity of a purified virus product. Such a process may be based on analysis of a purified virus using size exclusion chromatography coupled to high performance liquid chromatography (HPLC). As a result, we have developed a size exclusion chromatography HPLC method to evaluate the purities and quantity of adenovirus product previously purified by ion exchange chromatography.

### SUMMARY OF THE INVENTION

The present invention concerns methods for evaluating a purified virus sample. Often viruses are evaluated for purity using the same chromatographic medium used for the virus purification process. Such practice may lead to inaccurate measurements of purity or quality or quantity of a virus sample, as impurities or contaminants capable of passing through the chromatographic medium used to purify the virus may also pass through the same medium used to evaluate the aforementioned virus properties. The inventors have developed methods of evaluating various attributes of a previously purified virus sample by subjecting the virus sample to high performance size exclusion chromatography, also known as high performance liquid chromatography coupled to a size exclusion chromatographic medium.

Certain embodiments of the invention relate to methods of evaluating a virus sample, previously purified by means other than size exclusion chromatography, by subjecting the virus sample to high performance size exclusion chromatography and assessing the eluate from the high performance size exclusion chromatography by spectroscopy. Typically, such spectroscopy will result in the generation of a graphical representation of peaks via a computer monitor or printout. The assessment of the eluate by spectroscopy allows for evaluating the eluate by one or more of: 1) quantifying the virus sample is achieved by determining the area of the virus containing peak or peaks, 2) assessing the quality or “qualifying” the virus sample is achieved by assessing the symmetry of the virus containing peak or peaks, and/or 3) assessing the purity of the virus sample is achieved by dividing the area of the virus containing peaks by the area of all peaks of the graphical representation.

In certain embodiments the size exclusion chromatographic medium used in high performance size exclusion chromatography is Bio-Sep-SEC-S3000, Toyopearl HW-40C, Toyopearl HW40F G6000PWXL, Uniflow™, Superflow™, and Ultraflow™, Shodex™, Bio-Sil™, Bio-Gel™, SB-806, SEPHACRYL S400 HR, SEPHACRYL S-500 HR, SEPHACRYL S-1000 SF, SEPHADEX G-200, SEPHAROSE CL-2B, SUPERDEX 200 prep grade, SUPEROSE 6 prep grade, TSK 6000PWXL and ULTRAHYDROGEL 2000. In particular embodiments, the size exclusion chromatographic medium is Bio-Sep-SEC-S3000. In certain embodiments, the size exclusion chromatographic medium may be described in terms of pore size. In particular embodiments the pore size is between about 5 nm and about 100 nm. In specific embodiments the pore size of the chromatographic medium is about 29 nm.

When assessing the eluate of a virus sample subjected to high performance size exclusion chromatography, any form of spectroscopy is contemplated. Such forms of spectroscopy include, but are not limited to: refractive index detectors, wavelength absorbance detectors, diode array detectors, chiral detectors, chemiluminescence detectors, circular dichroism detectors, light scattering detectors and fluorescence emission detectors. In certain embodiments the spectroscopy method is wavelength absorbance, refractive index, fluorescence emission or light scattering.

In particular embodiments, when the spectroscopy method is wavelength absorbance, the wavelength is about 215 nm to about 280 nm. In still other embodiments, the wavelength is about 215 nm.

In the embodiments of the present invention the virus sample is previously purified by a means other than size exclusion chromatography. In certain embodiments the previous method of purification is ultracentrifugation, ion exchange chromatography, immobilized metal affinity chromatography, sulfated affinity chromatography, immunoaffinity chromatography, heparin affinity chromatography, hydrophobic interaction chromatography, hydroxyapatite chromatography or combinations thereof. In preferred embodiments, the virus sample is previously purified by ion exchange chromatography. In still more preferred embodiments the ion exchange chromatography is anion exchange chromatography. In particular embodiments the chromatographic medium used in the anion exchange chromatography is Amersham Biosciences Source 15Q.

In certain embodiments the virus sample which has been previously purified by means other than size exclusion chromatography will be stored for a period of time prior to evaluation of the virus sample according to the methods of the present invention. In certain embodiments the virus sample previously purified by means other than size exclusion chromatography will be stored for a period between 1, 2, 3, 4, 5 or 6 days, or 1, 2 or three weeks, or 1, 2, 3, 4, 5, 6, 7, 8, 9, 10 or 11 months or 1 to 2 years prior to determining the quantity, quality and purity of the virus sample according to the methods of the present invention. It is also contemplated that the virus sample may be stored for any combination of time intervals between one day and two years prior to determining the quantity, quality and purity of the virus sample according to the methods of the present invention. For example the virus sample may be stored for a time period of 2 months, three weeks and 5 days or any other conceivable interval between one day and two years prior to determining the quantity, quality and purity of the virus sample according to the methods of the present invention.

In other embodiments of the invention, the virus sample will be an adenovirus sample, an adeno-associated virus sample, a retrovirus sample, a herpesvirus sample, a pox virus sample, a picornavirus sample, or an alphavirus sample. In preferred embodiments the virus sample is an adenovirus sample.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 Adenovirus Size Exclusion Chromatography/HPLC chromatogram. 200 μl sample injection. Sample diluted 1:5 with running buffer (0.1 M phosphate mobile phase buffer. Chromatogram depicts 98.8% purity.

FIG. 2A Chromatogram of Adenovirus Size Exclusion Chromatography/HPLC. Sample collection took place at 5 ml intervals at 5-16 minutes post injection.

FIG. 2B SDS-Page analysis of fractions collected. Arrows indicate adenovirus fiber protein and 293 HCP.

FIG. 2C Western Blot analysis using anti-Ad-5 polyclonal antibody.

FIG. 3 Correlation between A215 nm area and viral particle concentration.

FIG. 4A Chromatogram of Adenovirus Size Exclusion Chromatography/HPLC of denatured adenovirus sample. Sample collection took place at 1 ml intervals starting at 5 ml post injection.

FIG. 4B SDS-PAGE analysis of fractions collected and Western Blot analysis using anti-Ad-5 polyclonal antibody. Arrows indicate adenoviral hexon, penton and fiber proteins.

### DESCRIPTION OF THE ILLUSTRATIVE EMBODIMENTS

The present invention involves novel methods of assessing the purity or stability of a virus sample or quantifying the number of virus particles in a virus sample. Contaminants arising out of the process for the production of adenovirus e.g., BSA and other proteins from the media, as well as host cell proteins, must be removed from an adenoviral preparation before such a preparation can be used in clinical applications. Accordingly, for such clinical applications it is useful, if not necessary to possess an accurate assessment of adenovirus to be used in such applications. For example it is often desirable to assess the quality, quantity or purity of a given adenovirus sample. Currently, adenovirus product is purified through ion-exchange (IEX) chromatography. The purified product is subsequently assessed for purity using ion exchange HPLC (IEX-HPLC). However, since adenovirus product is not likely to detect all impurities that are not removed through the purification process. While IEX-HPLC separation is based on differences in molecule charge, a size exclusion chromatography HPLC method (SEC-HPLC) which is based on differences in molecule size for separation, is expected to better detect the presence of residual impurities in the adenovirus product and provide additional purity information. Moreover, storage of adenoviral samples may result in some degradation. Such degradation may reduce the effectiveness of therapeutic dosages of such vectors. Thus, methods for determining the purity, quality or quantity of a given virus sample are desirable.

The present invention involves novel methods of assessing the purity or quality or quantity of a virus sample. This process is based on obtaining a previously purified virus sample and subjecting a portion of this purified virus sample to a chromatographic medium coupled with HPLC, wherein the chromatographic medium is not the same method which was used in the original virus purification method.

The virus, for example, may have been originally purified via ion exchange chromatography. Subsequently, the a portion of the virus may be subjected to, for example, SEC-HPLC, wherein the virus is analyzed by a detection method such as wavelength absorbance, and a graphical representation of the virus peak and any contaminating peaks may be produced via a computer or other electronic source. The method of analyzing the virus may be by any number of detection methods as described below, and need not necessarily be via wavelength absorbance. Once a graphical representation is produced, the quantity, quality or purity of the virus may be determined.

One such method of determining the quantity of the virus is to determine the area in the graphical representation that corresponds to the known virus. This could be compared to a standard curve of known virus quantities in order to determine the quantity of the purified virus sample. A graphical representation of the virus peak and any contaminating peaks may also be used to determine the quality and purity of the virus sample. For example, determining the purity of a virus sample could be performed by dividing the area of all peaks by the area of the virus peak. Determining the quality of the virus sample may be accomplished by evaluating the symmetry of the virus peak, with a greater symmetry of the virus peak indicating a higher quality of the virus sample and subsequently less degradation, which might be observed after mistreatment of the originally purified virus sample or due to long term storage. One such method for measuring the symmetry of the virus sample may be to bisect the virus peak with a vertical axis at the highest point, followed by determining the ratio of the area of one half of the bisected virus peak with the area of the other half of the virus peak.

A. Chromatographic Techniques

In certain embodiments of the invention, it will be necessary to evaluate aspects of a previously purified virus sample, such as a previously purified adenovirus sample. The virus sample may have been previously purified by chromatographic methods. If a previously purified virus sample is evaluated for quantity, quality or purity, the use of a different chromatographic medium will be advantageous. A virus product purified by the same type of chromatographic medium used in the later evaluation of the virus would likely not allow detection of residual impurities in the virus sample.

In certain embodiments of the invention, it will be desirable to assess the purity of a sample of a given virus, for example, an adenovirus sample. Purification techniques are well known to those of skill in the art. These techniques tend to involve the fractionation of the cellular lysate to separate the virus particles from other components of the mixture. Having separated viral particles from the other components, the virus may be purified using chromatographic and electrophoretic techniques to achieve complete purification. In certain embodiments of the invention, it will be desirable to assess the purity of a sample of a given virus, for example, an adenovirus sample. Analytical methods particularly suited to assessing the purity of viral particles of the present invention include ion-exchange chromatography (IEX), size exclusion chromatography (SEC); polyacrylamide gel electrophoresis (SDS/PAGE) and HPLC.

Various methods for quantifying the degree of purification of a virus will be known to those of skill in the art in light of the present disclosure. These include, for example, determining the relative peaks from HPLC analysis of the virus or assessing the amount of polypeptides within a fraction by SDS/PAGE analysis. One such method to assess the purity of a virus sample is to calculate the specific activity of the fraction, to compare it to the specific activity of the initial extract, and to thus calculate the degree of purity, herein assessed by a “-fold purification number”. However, a preferred method for assessing the purity of a fraction is to first subject a virus lysate to IEX followed subjecting the same sample to SEC coupled with HPLC. The actual units used to represent the purity or amount of activity will, of course, be dependent upon the particular assay technique chosen to follow the purification and whether or not the expressed virus exhibits a detectable activity.

There is no general requirement that the adenovirus, always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

1. High Performance Liquid Chromatography

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample can be low because the bands are so narrow that there is very little dilution of the sample.

Such high performance liquid chromatography may be combined with other chromatographic mediums. Examples of such combinatorial approaches include, but are not limited to: high performance size exclusion chromatography, high performance ion exchange chromatography, high performance immunoaffinity chromatography, high performance heparin affinity chromatography, high performance hydrophobic interaction chromatography, or high performance hydroxyapatite chromatography.

a. Liquid Chromatographic Detection Methods

Numerous methods of detecting substances via HPLC are known in the art. These methods of detection are based on the art of spectroscopy. Spectroscopy as defined herein includes the study of matter and its properties by investigating light, sound, or particles that are emitted, absorbed or scattered by the matter under investigation. Such spectroscopy methods include refractive index detectors, wavelength absorbance detectors, diode array detectors, chiral detectors, chemiluminescence detectors, circular dichroism detectors, light scattering detectors and fluorescence detectors. Among those methods which may be especially suitable for detecting virus samples include refractive index detectors, wavelength absorbance detectors, chemiluminescence detectors, light scattering detectors and fluorescence detectors. Examples of fluorescence emission detection used analysis of virus samples can be found in U.S. Pat. No. 6,630,299, herein incorporated by reference. Examples of light scattering detection used for analysis of virus samples can be found in U.S. Pat. No. 6,316,185, herein incorporated by reference. Examples of refractive index detection used for analysis of protein samples can be found in U.S. Pat. No. 5,491,096, herein incorporated by reference. Examples of chemiluminescence detection use for analysis of organisms can be found in U.S. Pat. No. 5,902,722. Examples of wavelength absorption detection used for analysis of virus samples can be found throughout the specification.

Typically, such detection methods will involve computer analysis to generate a graphical representation of peaks associated with virus samples and peaks associated with contaminants and other proteins. Persons of skill in the art will be familiar with the expected virus peak of the graphical representation.

While not intending to be restricted to a particular method of determining the quantity, quality or purity of a virus sample, one such method may include generating a standard curve of virus peak areas of known virus quantities and comparing the area of the virus peak of the analyzed virus to that of a standard curve of known virus concentrations. One method of the present invention contemplated by the inventors for measuring the purity of the virus composition involves dividing the area of the virus peak by the area of all peaks of the graphical representation. Symmetry of the virus peak may be a useful tool in the evaluation of the quality of the previously purified virus sample. For example, a purified virus sample which has been stored improperly or for a certain length of time may experience degradation of the product to be administered to a subject. The inventors have found that a symmetrical virus peak is often indicative of a product with little or no degradation, whereas a virus an asymmetrical virus peak may be indicative of virus degradation. One such method for determining symmetry of a virus peak may be to bisect the peak along an axis which crosses the apex of the virus peak. Symmetry could then be determined by determining the ratio of the area of one side of the virus peak by the area of the other side of the virus peak.

The following non-limiting examples of chromatographic mediums may be employed in the method of the present invention so long as the chromatographic medium coupled with HPLC, is not the same method or medium used to initially purify the virus sample.

2. Ion Exchange Chromatography

Ion-exchange chromatography relies on the affinity of a substance for the exchanger, which affinity depends on both the electrical properties of the material and the relative affinity of other charged substances in the solvent. In the process of ion-exchange chromatography, bound material can be eluted by changing the pH, thus altering the charge of the material, or by adding competing materials, for example salts. The conditions for release vary with each bound molecular species because different substances have different electrical properties. In general, to obtain optimal separation, the methods of choice are either continuous ionic strength gradient elution or stepwise elution. (A gradient of pH alone is not often used because it is difficult to set up a pH gradient without simultaneously increasing ionic strength.) In anion-exchange chromatography, often either pH and ionic strength are gradually increased or ionic strength alone is increased. Alternatively, if one chooses cation-exchange chromatography, often both pH and ionic strength are increased. The specific choice of the elution procedure is often a result of considerations of stability. For example, for unstable materials, it is best to maintain fairly constant pH.

An ion exchanger is a solid that has chemically bound charged groups to which ions are electrostatically bound. Thus an ion-exchanger is able to exchange these chemically bound charged groups for ions in an aqueous solution. An ion exchanger solid media can be employed in column chromatography to separate molecules or virus particles from contaminants according to charge.

The principle of ion-exchange chromatography is that charged molecules adsorb to ion exchangers reversibly so that molecules can be bound or eluted by changing the ionic environment. Separation on ion exchangers is usually accomplished in two stages: first, the substances to be separated are bound to the exchanger, using conditions that give stable and tight binding; then the column is eluted with buffers of different pH, ionic strength, or composition and the components of the buffer compete with the bound material for the binding sites.

An ion exchanger is usually a three-dimensional network or matrix that contains covalently linked charged groups. If a group is negatively charged, it will exchange positive ions and is a cation exchanger. A typical group used in cation exchangers is the sulfonic group, So₃⁻. If an H⁺ is bound to the group, the exchanger is said to be in the acid form; it can, for example, exchange on H⁺ for one Na⁺ or two H⁺ for one Ca⁺. The sulfonic acid group is a strongly acidic cation exchanger. Other commonly used groups are phenolic hydroxyl and carboxyl, both weakly acidic cation exchangers. If the charged group is positive, for example, a quaternary amino group, it is a strongly basic anion exchanger. The most common weakly basic anion exchangers are aromatic or aliphatic amino groups.

The matrix can be made of various material. Commonly used materials are dextran, cellulose, agarose and copolymers of styrene and vinylbenzene in which the divinylbenzene both cross-links the polystyrene strands and contains the charged groups.

The total capacity of an ion exchanger measures its ability to take up exchangeable groups per milligram of dry weight. This number is supplied by the manufacturer and is important because, if the capacity is exceeded, ions will pass through the column without binding.

3. Affinity Chromatography

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The support matrices commonly used in association with protein-binding ligands employed in affinity chromatography include, for example, polymers and copolymers of agarose, dextrans and amides, especially acrylamide, or glass beads or nylon matrices. Exemplary affinity chromatographic techniques are discussed in further detail below.

a. Immobilized Metal Affinity Chromatography

Immobilized metal affinity chromatography (IMAC), also known as metal chelate affinity chromatography (MCAC), is used primarily in the purification of polyhistidine tagged recombinant proteins. This is achieved by using the natural tendency of histidine to chelate divalent metals. Placing the metal ion on a chromatographic support allows purification of the histidine tagged proteins. This is a highly efficient method that has been employed by those of skill in the art for a variety of protein purification methods.

The high efficiency of the IMAC method is based on the interaction of a covalently bound chelating ligand immobilized on a chromatographic support with histidine-containing proteins. In this method, the metal ion must have a high affinity for the support. Commonly used as the supporting matrix are iminodiacetic acid derivatives.

The most common chelating group used in this technique is iminodiacetic acid (IDA). It is coupled to a matrix such as Sepharose M 6B, via a long hydrophilic spacer arm. The spacer arm ensures that the chelating metal is fully accessible to all available binding sites on a protein. Affiland (Ans-Liege, Belgium) is one exemplary commercial source of immobilized iminodiacetic acid (IDA), nitrilotriacetic acid (NTA) and a pentadentate chelator (PDC) ligand for IMAC (see world wide web at affiland.com/imac.htm). Briefly, immobilized IDA is a tridentate ligand at physiological pH, NTA is a pentadentate ligand at basic pH and a tridentate ligand at pH 8.0. In the presence of the electron donor cross-linkers, immobilized IDA forms octahedral complexes with polyvalent metal ions including Cu²⁺, Zn²⁺, Ni²⁺ and Co²⁺. This column has a selective binding for histidine-containing proteins. The elution of histidine-containing proteins of interest uses a high concentration of Imidazole.

The IDA matrix is supplied bound to a number of underlying matrices e.g., Sepharose, and the like. The ISA-matrix is degassed and then applied to a column and washed with 10 volumes of distilled water. The bivalent or trivalent cation is then applied to the washed matrix in a distilled water at a concentration 5 mg/ml in distilled water, at a flow rate of 50 ml/cm²/hour, until saturation. The metal chelate affinity matrix is then equilibrated with an appropriate buffer e.g., Tris 50 mM, AcOH pH 8.0. The equilibrated column is then ready for use. Similar procedures are described for the NTA and the PDC matrices on the world wide web at affiland.com/imac/nta.htm and affiland.com/imac/pdc.htm, respectively). Fractogel™ EMD chelate iminodiacetic acid is an IMAC matrix supplied by VWR International, Merck (Poole, Dorset, U.K.). TALON™ resin is a durable IMAC resin that uses cobalt ions for purifying recombinant polyhistidine-tagged proteins (Clontech, Palo Alto, Calif.).

Another common chelating group for IMAC applications is tris(carboxymethyl)-ethylenediamine (TED). TED gels show stronger retention of metal ions and weaker retention of proteins relative as compared to IDA-based matrices. TED matrices form a complex (single coordination site) whereas IDA matrices form a chelate (multiple coordination sites). The most commonly used metals for IMAC are zinc and copper; however, nickel cobalt, and calcium have also been used successfully.

Suitable immobilized metal affinity media include, Chelating Sepharose Fast Flow (Amersham Biosciences AB, Uppsala Sweden), HiTrap Chelating Media (Sigma-Aldrich, St. Louis, Mo.), and TSKgel Chelate-5PW (Sigma-Aldrich, St. Louis, Mo.).

b. Sulfated Affinity Chromatography

Sulfated affinity chromatography uses oligosaccharide (generally cellulose) resins as support matrices. These resins are derivatized with a sulfate compound. The sulfated affinity chromatographic medium attracts certain surface proteins or contaminants that are attracted to sulfate. U.S. Pat. No. 5,447,859, describes the use of sulfated affinity media in the purification of viruses. Suitable sulfated affinity media include, Matrex Cellufine Sulfate Affinity Media (Millipore, Bedford, Mass.), and Sterogene Sulfated Hi Flow (Carlsbad, Calif.)

c. Immunoaffinity Chromatography

Immunoaffinity column chromatography involves the preparation of a column media in which the matrix of the chromatographic medium is linked to an antibody or an antigen, that can specifically bind the target species (i.e., antigen or antibody, respectively) from a complex mixture. Immunoaffinity chromatography is specific for the species of interest being isolated and may be performed under mild conditions. Immunoaffinity purification techniques are well known in the art (see, Harlow, et al., 1988).

d. Heparin Affinity Chromatography

Heparin affinity media is another commonly used affinity chromatography. Heparin has two properties that facilitate its use in chromatographic techniques. It can act as an affinity ligand, for example, in its interaction with coagulation factors, or heparin can function as a high capacity cation exchanger, due to its anionic sulfate groups. Gradient elution with salt is most commonly used in both cases to elute the bound species from the column. Suitable heparin affinity media include but are not limited to Heparin Sepharose 6 Fast Flow (Amersham Biosciences AB, Uppsala Sweden), HiTrap Heparin HP (Amersham Biosciences AB, Uppsala Sweden), and Cellufine Heparin (Millipore, Bedford, Mass.).

4. Hydrophobic Interaction Chromatography

Hydrophobic Interaction Chromatography (HIC) is based on hydrophobic attraction between the stationary phase and the protein molecules. The stationary phase consists of small non-polar groups (butyl, octyl or phenyl) attached to a hydrophilic polymer backbone (cross-linked dextran or agarose, for example). Separations by HIC are often designed using nearly opposite conditions to those used in ion exchange chromatography. Binding of the proteins is often carried out at high salt concentration. Some proteins may precipitate at this high ionic strength, thus necessitating removal by centrifugation prior to loading the protein mixture onto the column. Selective elution of bound proteins is then carried out by applying a decreasing salt gradient.

Any suitable chromatographic material can be used. For example, a variety of different chromatographic materials supports are commercially available which have hydrophobic ligands attached to a chromatographic support. For example, the ligands may have an alkyl chain ranging from about two to twenty or more carbons in length. These ligands may be branched, linear, or contain carbon rings, such as phenyl rings. Increasing chain length typically results in a chromatographic medium with greater hydrophobicity. Commonly used ligands are phenyl-, butyl-, and octyl-residues. Commercially available hydrophobic interaction chromatographic materials include, but are not limited to: POROS HP2®, POROS PE® and POROS ET® (Applied Biosystems, Foster City, Calif.); Bio-Rad Macro-Prep HIC Supports, Bio-Rad Methyl HIC support, Bio-Rad-t-butyl HIC support, Bio-rad Econo column butyl-650m (Bio-Rad, Hercules, Calif.) TosoHaas TSK-GELO and TosoHaas TOKYOPEARL (Tosh Bioscience, Montgomeryville, Pa.); Fractogel® EMD Propyl (S) and EMD Phenyl I (S) (Merck, Darmstadt, Germany); IEC PH-814 (Phenomenex, Torrence, Calif.) and HiPrep™ 16/10 Phenyl, HiPrep™ 16/1-Butyl, HiPrep™ 16/10 Octyl, HiLoad Phenyl-Sepharose FF and HiLoad Butyl-Sepharose FF (GE Helthcare, Little Chalfont, UK).

A further detailed description of the general principles of hydrophobic interaction chromatography media may be found in U.S. Pat. No. 3,917,527 and in U.S. Pat. No. 4,000,098, each incorporated by reference herein. The application of HIC to the purification of specific proteins is exemplified by reference to the following disclosures, each of which is herein incorporated by reference: human growth hormone (U.S. Pat. No. 4,332,717), toxin conjugates (U.S. Pat. No. 4,771,128), antihemolytic factor (U.S. Pat. No. 4,743,680), tumor necrosis factor (U.S. Pat. No. 4,894,439), interleukin-2 (U.S. Pat. No. 4,908,434), human lymphotoxin (U.S. Pat. No. 4,920,196) and lysozyme species (Fausnaugh and Regnier, 1986) and soluble complement receptors (U.S. Pat. No. 5,252,216).

5. Hydroxyapatite Chromatography

Hydroxyapatite chromatography is a method of purifying proteins that utilizes an insoluble hydroxylated calcium phosphate which forms both the matrix and ligand. Functional groups consist of pairs of positively charged calcium ions (C-sites) and clusters of negatively charged phosphate groups (P-sites).

Various hydroxyapatite chromatographic resins are available commercially, and any available form of the material can be used in the practice of this invention. In one embodiment of the invention, the hydroxyapatite is in a crystalline form. Hydroxyapatites for use in this invention may be those that are agglomerated to form particles and sintered at high temperatures into a stable porous ceramic mass.

A number of chromatographic supports may be employed in the preparation of hydroxyapatite chromatography columns, the most extensively used are Type I and Type II hydroxyapatite. Type I has a high protein binding capacity and better capacity for acidic proteins. Type II, however, has a lower protein binding capacity, but has better resolution of nucleic acids and certain proteins. The Type II material also has a very low affinity for albumin and is especially suitable for the purification of many species and classes of immunoglobulins. The choice of an application appropriate hydroxyapatite may be determined by those of skill in the art. Commercially available hydroxyapatite chromatographic materials include, but are not limited to: CHT™ Ceramic Hydroxyapatite, Type I, (20, 40 or 80 μm) and CHT™ Ceramic Hydroxyapatite, Type II, (20, 40 or 80 μm) (Bio-Rad, Hercules, Calif.) and HA-Ultrogel® (Sigma-Aldrich, St. Louis, Mo.).

6. Size Exclusion Chromatography

Size exclusion chromatography, otherwise known as gel filtration or gel permeation chromatography, relies on the penetration of macromolecules in a mobile phase into the pores of stationary phase particles. Differential penetration of the macromolecules is a function of the hydrodynamic volume of the particles. Size exclusion media exclude larger molecules from the interior of the particles while the smaller molecules are accessible to this volume. The order of elution can be predicted by the size of the protein as a linear relationship exists between elution volume and the log of the molecular weight of the protein being eluted.

Media appropriate for size-exclusion chromatography of adenoviruses include but are not limited to such resins as Toyopearl HW-40C, Toyopearl HW40F and G6000PWXL (TosoHaas, Montgomeryville, Pa.); Uniflow™, Superflow™, and Ultraflow™ (Sterogene, Carlsbad, Calif.); Shodex™ (Thomson Instruments, Chantilly, Va.); Bio-Sil™ and Bio-Gel™ (Bio-Rad, Hercules, Calif.); SB-806 (Alltech, Nicholasville, Ken.); SEPHACRYL S400 HR, SEPHACRYL S-500 HR, SEPHACRYL S-1000 SF, SEPHADEX G-200, SEPHAROSE CL-2B; SUPERDEX 200 prep grade, SUPEROSE 6 prep grade (Pfizer, New York, N.Y.); TSK 6000PWXL (Bodman, Aston, Pa.), and ULTRAHYDROGEL 2000 (Waters, Milford, Mass.). A particularly useful size exclusion chromatography column of the present invention is the Bio-Sep-SEC-S3000 (Phenomenex, Torrance, Calif.).

In particular, size exclusion chromatographic media may be described in terms of pore size of the material. Table 1 provides a non-limiting example of suitable size exclusion chromatographic mediums and their corresponding pore size which may be appropriate for size exclusion chromatographic techniques involving adenoviruses.

TABLE 1

| | Pore Size |
|---|---|
| Column Name | (nm) |
| Bio-Sep-SEC-S2000 | 14.5 |
| Bio-Sep-SEC-S3000 | 29 |
| Bio-Sep-SEC-S4000 | 50 |
| Toyopearl HW-40 | 5 |
| Toyopearl HW-50 | 12.5 |
| Toyopearl HW-55 | 50 |
| Toyopearl HW-65 | 100 |
| TSK-GEL G2000SW | 12.5 |
| TSK-GEL G2000SWXL | 12.5 |
| TSK-GEL G3000SW | 25 |
| TSK-GEL G3000SWXL | 25 |
| TSK-GEL G4000SW | 45 |
| TSK-GEL G4000SWXL | 45 |
| TSK-GEL G4000PWXL | 50 |
| TSK-GEL G5000PW | 100 |

B. Methods of Cell Culture and Virus Propagation

Various embodiments of the present invention involve methods for assaying the purity or quality of or quantifying the number of viral particles in a virus sample. In some embodiments the virus product is produced via the use of a host cell. A “host cell” is defined as a cell that is capable of supporting replication of adenovirus. Any cell type for use as a host cell is contemplated by the present invention, as long as the cell is capable of supporting replication of adenovirus. For example, the host cells may be HEK293, PER.C6, 911, or IT293SF cells. One of skill in the art would be familiar with the wide range of host cells that are available for use in methods for producing an adenovirus.

1. Cells

The generation and propagation of adenoviral vectors, such as replication defective adenoviral vectors often depends on a unique helper cell line, designated 293, which was transformed from human embryonic kidney cells by Adenovirus serotype 5 (AdS) DNA fragments and constitutively expresses E1 proteins (Graham et al., 1977). Since the E3 region is dispensable from the Ad genome (Jones and Shenk, 1978), the current Ad vectors, with the help of 293 cells, carry foreign DNA in either the E1, the E3 or both regions (Graham and Prevec, 1991; Bett et al., 1994). These cells lines are capable of supporting replication of adenovirus recombinant vectors and helper viruses having defects in certain adenoviral genes, i.e., are “permissive” for growth of these viruses and vectors. The recombinant cell also is referred to as a helper cell because of the ability to complement defects in, and support replication of, replication-incompetent adenoviral vectors. The prototype for an adenoviral helper cell is the 293 cell line, which contains the adenoviral E1 region. 293 cells support the replication of adenoviral vectors lacking E1 functions by providing in trans the E1-active elements necessary for replication. Other cell lines which also support the growth of adenoviruses lacking E1 function include PER.C6 (IntroGene, NL), 911 (IntroGene, NL), and IT293SF.

Helper cells are derived from a mammalian cell and, preferably, from a primate cell such as human embryonic kidney cell. Although various primate cells are preferred and human or even human embryonic kidney cells are most preferred, any type of cell that is capable of supporting replication of the virus would be acceptable in the practice of the invention. Other cell types might include, but are not limited to Vero cells, HeLa cells or any eukaryotic cells for which tissue culture techniques are established as long as the cells are adenovirus permissive. The term “adenovirus permissive” means that the adenovirus or adenoviral vector is able to complete the entire intracellular virus life cycle within the cellular environment.

The helper cell may be derived from an existing cell line, e.g., from a 293 cell line, or developed de novo. Such helper cells express the adenoviral genes necessary to complement in trans deletions in an adenoviral genome or which support replication of an otherwise defective adenoviral vector, such as the E1, E2, E4, E5 and late functions. A particular portion of the adenovirus genome, the E1 region, has already been used to generate complementing cell lines. Whether integrated or episomal, portions of the adenovirus genome lacking a viral origin of replication, when introduced into a cell line, will not replicate even when the cell is superinfected with wild-type adenovirus. In addition, because the transcription of the major late unit is after viral DNA replication, the late functions of adenovirus cannot be expressed sufficiently from a cell line. Thus, the E2 regions, which overlap with late functions (L1-5), will be provided by helper viruses and not by the cell line. Typically, a cell line according to the present invention will express E1 and/or E4.

As used herein, the term “recombinant” cell is intended to refer to a cell into which a gene, such as a gene from the adenoviral genome or from another cell, has been introduced. Therefore, recombinant cells are distinguishable from naturally-occurring cells which do not contain a recombinantly-introduced gene. Recombinant cells are thus cells having a gene or genes introduced through “the hand of man.”

Replication is determined by contacting a layer of uninfected cells, or cells infected with one or more helper viruses, with virus particles, followed by incubation of the cells. The formation of viral plaques, or cell free areas in the cell layer, is the result of cell lysis caused by the expression of certain viral products. Cell lysis is indicative of viral replication.

Examples of other useful mammalian cell lines that may be used with a replication competent virus or converted into complementing host cells for use with replication deficient virus are Vero and HeLa cells and cell lines of Chinese hamster ovary, W138, BHK, COS-7, HepG2, 3T3, RIN, MDCK and A549 cells.

2. Growth in Selection Media

In certain embodiments, it may be useful to employ selection systems that preclude growth of undesirable cells. This may be accomplished by virtue of permanently transforming a cell line with a selectable marker or by transducing or infecting a cell line with a viral vector that encodes a selectable marker. In either situation, culture of the transformed/transduced cell with an appropriate drug or selective compound will result in the enhancement, in the cell population, of those cells carrying the marker.

Examples of markers include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside G418; and hygro, that confers resistance to hygromycin.

3. Growth in Serum Weaning

Serum weaning adaptation of anchorage-dependent cells into serum-free suspension cultures have been used for the production of recombinant proteins (Berg, 1993) and viral vaccines (Perrin, 1995). Gilbert reported the adaptation of 293A cells into serum-free suspension cultures for adenovirus and recombinant protein production (Gilbert, 1996). A similar adaptation method had been used for the adaptation of A549 cells into serum-free suspension culture for adenovirus production (Morris et al., 1996). Cell-specific virus yields in the adapted suspension cells, however, are about 5-10-fold lower than those achieved in the parental attached cells.

4. Adaptation of Cells for Suspension Culture

Various methods have been used to adapt cells into suspension cultures. For example, in the present invention, HeLa cells adapted for growth in serum-free conditions were adapted into a suspension culture. The cells were transferred in a serum-free 250 ml spinner suspension culture (100 ml working volume) for the suspension culture at an initial cell density of between about 1.18×10⁵ cells/ml and about 5.22×10⁵ cells/ml. The media may be supplemented with heparin to prevent aggregation of cells. This cell culture system allows for some increase of cell density while cell viability is maintained. Once the cells are growing in culture, they are passaged approximately 7 times in the spinner flasks.

5. Cell Culture Systems

Virus samples of the methods of the present invention may be propagated via host cells grown in bioreactors. Growing cells according to the present invention in a bioreactor allows for large scale production of fully biologically-active cells capable of being infected by the adenoviral vectors of the present invention.

As used herein, a “bioreactor” refers to any apparatus that can be used for the purpose of culturing cells. Growing cells in a bioreactor allows for large scale production of fully biologically active cells capable of being infected by the adenoviral vectors of the present invention. Bioreactors have been widely used for the production of biological products from both suspension and anchorage dependent animal cell cultures. For example, the most widely used producer cells for adenoviral vector production are anchorage dependent human embryonic kidney cells (293 cells). Microcarrier cell culture in stirred tank bioreactor provides very high volume-specific culture surface area and has been used for the production of viral vaccines (Griffiths, 1986). Furthermore, stirred tank bioreactors have industrially proven to be scaleable. The multiplate CellCube™ cell culture system manufactured by Corning-Costar also offers a very high volume specific surface area. Cells grow on both sides of the culture plates hermetically sealed together in the shape of a compact cube. Unlike stirred tank bioreactors, the CellCube™ culture unit is disposable.

Another example of a bioreactor that may be employed to generate viral samples contemplated in the present invention is a Wave Bioreactor®. The Wave Bioreactor® can be a Wave Biotech® model 20/50EH. According to a preferred aspect of the invention, the Wave Bioreactor® is used with serum-free media. As used herein, “media” and “medium” refers to any substance which can facilitate growth of host cells. According to one aspect of the present invention, the host cells are grown in media that is serum-free media. One example of a protein-free media is CD293. Another example of media that can support host cell growth is DMEM +2% FBS. One of skill in the art would understand that various components and agents can be added to the media to facilitate and control cell growth.

a. Anchorage-Dependent Versus Non-Anchorage-Dependent Cultures

Animal and human cells can be propagated in vitro in two modes: as non-anchorage dependent cells growing freely in suspension throughout the bulk of the culture; or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. Large scale suspension culture based on microbial (bacterial and yeast) fermentation technology has clear advantages for the manufacturing of mammalian cell products. The processes are relatively simple to operate and straightforward to scale up. Homogeneous conditions can be provided in the reactor which allows for precise monitoring and control of temperature, dissolved oxygen, and pH, and ensures that representative samples of the culture can be taken.

b. Reactors and Processes for Suspension

Two suspension culture reactor designs are most widely used in the industry due to their simplicity and robustness of operation—the stirred reactor and the airlift reactor. The stirred reactor design has successfully been used on a scale of 8000 liter capacity for the production of interferon (Phillips et al., 1985; Mizrahi, 1983). Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gasses and generates relatively low shear forces.

Most large-scale suspension cultures are operated as batch or fed-batch processes because they are the most straightforward to operate and scale up. However, continuous processes based on chemostat or perfusion principles are available.

A batch process is a closed system in which a typical growth profile is seen. A lag phase is followed by exponential, stationary and decline phases. In such a system, the environment is continuously changing as nutrients are depleted and metabolites accumulate. This makes analysis of factors influencing cell growth and productivity, and hence optimization of the process, a complex task. Productivity of a batch process may be increased by controlled feeding of key nutrients to prolong the growth cycle. Such a fed-batch process is still a closed system because cells, products and waste products are not removed.

In what is still a closed system, perfusion of fresh medium through the culture can be achieved by retaining the cells with a variety of devices (e.g. fine mesh spin filter, hollow fiber or flat plate membrane filters, settling tubes). Spin filter cultures can produce cell densities of approximately 5×10⁷ cells/ml. A true open system and the simplest perfusion process is the chemostat in which there is an inflow of medium and an outflow of cells and products. Culture medium is fed to the reactor at a predetermined and constant rate which maintains the dilution rate of the culture at a value less than the maximum specific growth rate of the cells (to prevent washout of the cell mass from the reactor). Culture fluid containing cells and cell products and byproducts is removed at the same rate.

c. Non-Perfused Attachment Systems

Traditionally, anchorage-dependent cell cultures are propagated on the bottom of small glass or plastic vessels. The restricted surface-to-volume ratio offered by classical and traditional techniques, suitable for the laboratory scale, has created a bottleneck in the production of cells and cell products on a large scale. In an attempt to provide systems that offer large accessible surfaces for cell growth in small culture volume, a number of techniques have been proposed: the roller bottle system, the stack plates propagator, the spiral film bottles, the hollow fiber system, the packed bed, the plate exchanger system, and the membrane tubing reel. Since these systems are non-homogeneous in their nature, and are sometimes based on multiple processes, they suffer from the following shortcomings—limited potential for scale-up, difficulties in taking cell samples, limited potential for measuring and controlling key process parameters and difficulty in maintaining homogeneous environmental conditions throughout the culture.

Despite these drawbacks, a commonly used process for large scale anchorage-dependent cell production is the roller bottle. Being little more than a large, differently shaped T-flask, simplicity of the system makes it very dependable and, hence, attractive. Fully automated robots are available that can handle thousands of roller bottles per day, thus eliminating the risk of contamination and inconsistency associated with the otherwise required intense human handling. With frequent media changes, roller bottle cultures can achieve cell densities of close to 0.5×10⁶ cells/cm² (corresponding to approximately 10⁹ cells/bottle or almost 10⁷ cells/ml of culture media).

d. Cultures on Microcarriers

In an effort to overcome the shortcomings of the traditional anchorage-dependent culture processes, van Wezel, (1967) developed the concept of the microcarrier culturing systems. In this system, cells are propagated on the surface of small solid particles suspended in the growth medium by slow agitation. Cells attach to the microcarriers and grow gradually to confluency on the microcarrier surface. In fact, this large scale culture system upgrades the attachment dependent culture from a single disc process to a unit process in which both monolayer and suspension culture have been brought together. Thus, combining the necessary surface for a cell to grow with the advantages of the homogeneous suspension culture increases production.

The advantages of microcarrier cultures over most other anchorage-dependent, large-scale cultivation methods are several fold. First, microcarrier cultures offer a high surface-to-volume ratio (variable by changing the carrier concentration) which leads to high cell density yields and a potential for obtaining highly concentrated cell products. Cell yields are up to 1-2×10⁷ cells/ml when cultures are propagated in a perfused reactor mode. Second, cells can be propagated in one unit process vessels instead of using many small low-productivity vessels (i.e., flasks or dishes). This results in far better nutrient utilization and a considerable saving of culture medium. Moreover, propagation in a single reactor leads to reduction in need for facility space and in the number of handling steps required per cell, thus reducing labor cost and risk of contamination. Third, the well-mixed and homogeneous microcarrier suspension culture makes it possible to monitor and control environmental conditions (e.g., pH, PO₂, and concentration of medium components), thus leading to more reproducible cell propagation and product recovery. Fourth, it is possible to take a representative sample for microscopic observation, chemical testing, or enumeration. Fifth, since microcarriers settle out of suspension quickly, use of a fed-batch process or harvesting of cells can be done relatively easily. Sixth, the mode of the anchorage-dependent culture propagation on the microcarriers makes it possible to use this system for other cellular manipulations, such as cell transfer without the use of proteolytic enzymes, cocultivation of cells, transplantation into animals, and perfusion of the culture using decanters, columns, fluidized beds, or hollow fibers for microcarrier retainment. Seventh, microcarrier cultures are relatively easily scaled up using conventional equipment used for cultivation of microbial and animal cells in suspension.

e. Microencapsulation of Mammalian Cells

One method which has shown to be particularly useful for culturing mammalian cells is microencapsulation. The mammalian cells are retained inside a semipermeable hydrogel membrane. A porous membrane is formed around the cells permitting the exchange of nutrients, gases, and metabolic products with the bulk medium surrounding the capsule. Several methods have been developed that are gentle, rapid and non-toxic and where the resulting membrane is sufficiently porous and strong to sustain the growing cell mass throughout the term of the culture. These methods are all based on soluble alginate gelled by droplet contact with a calcium-containing solution. U.S. Pat. No. 4,352,883, incorporated herein by reference,) describes cells concentrated in an approximately 1% solution of sodium alginate which are forced through a small orifice, forming droplets, and breaking free into an approximately 1% calcium chloride solution. The droplets are then cast in a layer of polyamino acid that ionically bonds to the surface alginate. Finally the alginate is reliquefied by treating the droplet in a chelating agent to remove the calcium ions. Other methods use cells in a calcium solution to be dropped into a alginate solution, thus creating a hollow alginate sphere. A similar approach involves cells in a chitosan solution dropped into alginate, also creating hollow spheres.

Microencapsulated cells are easily propagated in stirred tank reactors and, with beads sizes in the range of 150-1500 μm in diameter, are easily retained in a perfused reactor using a fine-meshed screen. The ratio of capsule volume to total media volume can be maintained from as dense as 1:2 to 1:10. With intracapsular cell densities of up to 108, the effective cell density in the culture is 1-5×10⁷.

The advantages of microencapsulation over other processes include the protection from the deleterious effects of shear stresses which occur from sparging and agitation, the ability to easily retain beads for the purpose of using perfused systems, scale up is relatively straightforward and the ability to use the beads for implantation.

The current invention includes cells which are anchorage-dependent in nature. HeLa cells, for example, are anchorage-dependent, and when grown in suspension, the cells will attach to each other and grow in clumps, eventually suffocating cells in the inner core of each clump as they reach a size that leaves the core cells unsustainable by the culture conditions.

6. Methods of Cell Harvest and Lysis

Adenoviral infection results in the lysis of cells being infected. Two different methods may be employed. One method is harvesting of the infected cells prior to lysis. The other method is harvesting of the cell supernatant after complete cell lysis by the produced virus. For the latter mode, longer incubation times are required to achieve complete cell lysis. Table 2 lists the most common methods that have been used for lysing cells after cell harvest.

TABLE 2

| Methods | Procedures | Comments |
|---|---|---|
| Freeze-thaw | Cycling between dry ice | Easy to carry out at lab scale. |
| | and 37° C. water bath | High cell lysis efficiency |
| | | Not scaleable |
| | | Not recommended for large |
| | | scale manufacturing |
| Solid Shear | French Press | Capital equipment investment |
| | Hughes Press | Virus containment concerns |
| | | Lack of experience |
| Detergent | Non-ionic detergent | Easy to carry out at both |
| lysis | solutions such as Tween, | lab and manufacturing scale |
| | Triton, NP-40, etc. | Wide variety of detergent |
| | | choices |
| | | Concerns of residual |
| | | detergent in finished |
| | | product |
| Hypotonic | water, citric buffer | Low lysis efficiency |
| solution | | |
| lysis | | |
| Liquid Shear | Homogenizer | Capital equipment investment |
| | Impinging Jet | Virus containment concerns |
| | Microfluidizer | Scaleability concerns |
| Sonication | Ultrasound | Capital equipment investment |
| | | Virus containment concerns |
| | | Noise pollution |
| | | Scaleability concern |

a. Detergents

Cells are bounded by membranes. In order to release components of the cell, it is necessary to break open the cells. The most advantageous way in which this can be accomplished, according to the present invention, is to solubilize the membranes with the use of detergents. Detergents are amphipathic molecules with an apolar end of aliphatic or aromatic nature and a polar end which may be charged or uncharged. Detergents are more hydrophilic than lipids and thus have greater water solubility than lipids. They allow for the dispersion of water insoluble compounds into aqueous media and are used to isolate and purify proteins in a native form.

Detergents can be denaturing or non-denaturing. The former can be anionic such as sodium dodecyl sulfate or cationic such as ethyl trimethyl ammonium bromide. These detergents totally disrupt membranes and denature the protein by breaking protein-protein interactions. Non denaturing detergents can be divided into non-anionic detergents such as Triton®X-100, bile salts such as cholates and zwitterionic detergents such as CHAPS. Zwitterionics contain both cationic and anion groups in the same molecule, the positive electric charge is neutralized by the negative charge on the same or adjacent molecule.

Denaturing agents such as SDS bind to proteins as monomers and the reaction is equilibrium driven until saturated. Thus, the free concentration of monomers determines the necessary detergent concentration. SDS binding is cooperative i.e. the binding of one molecule of SDS increase the probability of another molecule binding to that protein, and alters proteins into rods whose length is proportional to their molecular weight.

Non-denaturing agents such as Triton®X-100 do not bind to native conformations nor do they have a cooperative binding mechanism. These detergents have rigid and bulky apolar moieties that do not penetrate into water soluble proteins. They bind to the hydrophobic parts of proteins. Triton®X-100 and other polyoxyethylene nonanionic detergents are inefficient in breaking protein-protein interaction and can cause artifactual aggregations of protein. These detergents will, however, disrupt protein-lipid interactions but are much gentler and capable of maintaining the native form and functional capabilities of the proteins.

Detergent removal can be attempted in a number of ways. Dialysis works well with detergents that exist as monomers. Dialysis is somewhat ineffective with detergents that readily aggregate to form micelles because the micelles are too large to pass through dialysis. Ion exchange chromatography can be utilized to circumvent this problem. The disrupted protein solution is applied to an ion exchange chromatography column and the column is then washed with buffer minus detergent. The detergent will be removed as a result of the equilibration of the buffer with the detergent solution. Alternatively the protein solution may be passed through a density gradient. As the protein sediments through the gradients the detergent will come off due to the chemical potential.

Often a single detergent is not versatile enough for the solubilization and analysis of the milieu of proteins found in a cell. The proteins can be solubilized in one detergent and then placed in another suitable detergent for protein analysis. The protein detergent micelles formed in the first step should separate from pure detergent micelles. When these are added to an excess of the detergent for analysis, the protein is found in micelles with both detergents. Separation of the detergent-protein micelles can be accomplished with ion exchange or gel filtration chromatography, dialysis or buoyant density type separations.

Triton®X-Detergents: This family of detergents (Triton®X-100, X114 and NP-40) have the same basic characteristics but are different in their specific hydrophobic-hydrophilic nature. All of these heterogeneous detergents have a branched 8-carbon chain attached to an aromatic ring. This portion of the molecule contributes most of the hydrophobic nature of the detergent. Triton®X detergents are used to solubilize membrane proteins under non-denaturing conditions. The choice of detergent to solubilize proteins will depend on the hydrophobic nature of the protein to be solubilized. Hydrophobic proteins require hydrophobic detergents to effectively solubilize them.

Triton®X-100 and NP-40 are very similar in structure and hydrophobicity and are interchangeable in most applications including cell lysis, delipidation protein dissociation and membrane protein and lipid solubilization. Generally 2 mg detergent is used to solubilize 1 mg membrane protein or 10 mg detergent/1 mg of lipid membrane. Triton®X-114 is useful for separating hydrophobic from hydrophilic proteins.

Brij® Detergents: These are similar in structure to Triton®X detergents in that they have varying lengths of polyoxyethylene chains attached to a hydrophobic chain. However, unlike Triton®X detergents, the Brij® detergents do not have an aromatic ring and the length of the carbon chains can vary. The Brij® detergents are difficult to remove from solution using dialysis but may be removed by detergent removing gels. Brij®58 is most similar to Triton®X100 in its hydrophobic/hydrophilic characteristics. Brij®-35 is a commonly used detergent in HPLC applications.

Dializable Nonionic Detergents: η-Octyl-β-D-glucoside (octylglucopyranoside) and η-Octyl-β-D-thioglucoside (octylthioglucopyranoside, OTG) are nondenaturing nonionic detergents which are easily dialyzed from solution. These detergents are useful for solubilizing membrane proteins and have low UV absorbances at 280 nm. Octylglucoside has a high CMC of 23-25 mM and has been used at concentrations of 1.1-1.2% to solubilize membrane proteins.

Octylthioglucoside was first synthesized to offer an alternative to octylglucoside. Octylglucoside is expensive to manufacture and there are some inherent problems in biological systems because it can be hydrolyzed by β-glucosidase.

Tween® Detergents: The Tween® detergents are nondenaturing, nonionic detergents. They are polyoxyethylene sorbitan esters of fatty acids. Tween® 20 and Tween® 80 detergents are used as blocking agents in biochemical applications and are usually added to protein solutions to prevent nonspecific binding to hydrophobic materials such as plastics or nitrocellulose. They have been used as blocking agents in ELISA and blotting applications. Generally, these detergents are used at concentrations of 0.01-1.0% to prevent nonspecific binding to hydrophobic materials.

Tween® 20 and other nonionic detergents have been shown to remove some proteins from the surface of nitrocellulose. Tween® 80 has been used to solubilize membrane proteins, present nonspecific binding of protein to multiwell plastic tissue culture plates and to reduce nonspecific binding by serum proteins and biotinylated protein A to polystyrene plates in ELISA.

The difference between these detergents is the length of the fatty acid chain. Tween® 80 is derived from oleic acid with a C₁₈ chain while Tween® 20 is derived from lauric acid with a C₁₂ chain. The longer fatty acid chain makes the Tween® 80 detergent less hydrophilic than Tween® 20 detergent. Both detergents are very soluble in water.

The Tween® detergents are difficult to remove from solution by dialysis, but Tween® 20 can be removed by detergent removing gels. The polyoxyethylene chain found in these detergents makes them subject to oxidation (peroxide formation) as is true with the Triton® X and Brij® series detergents.

Zwitterionic Detergents: The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM). This detergent has low absorbances at 280 nm making it useful when protein monitoring at this wavelength is necessary. CHAPS is compatible with the BCA Protein Assay and can be removed from solution by detergent removing gel. Proteins can be iodinated in the presence of CHAPS

CHAPS has been successfully used to solubilize intrinsic membrane proteins and receptors and maintain the functional capability of the protein. When cytochrome P-450 is solubilized in either Triton® X-100 or sodium cholate aggregates are formed.

b. Non-Detergent Methods

Various non-detergent methods, though not preferred, may be employed in conjunction with other advantageous aspects of the present invention:

Freeze-Thaw: This has been a widely used technique for lysis cells in a gentle and effective manner. Cells are generally frozen rapidly in, for example, a dry ice/ethanol bath until completely frozen, then transferred to a 37° C. bath until completely thawed. This cycle is repeated a number of times to achieve complete cell lysis.

Sonication: High frequency ultrasonic oscillations have been found to be useful for cell disruption. The method by which ultrasonic waves break cells is not fully understood but it is known that high transient pressures are produced when suspensions are subjected to ultrasonic vibration. The main disadvantage with this technique is that considerable amounts of heat are generated. In order to minimize heat effects specifically designed glass vessels are used to hold the cell suspension. Such designs allow the suspension to circulate away from the ultrasonic probe to the outside of the vessel where it is cooled as the flask is suspended in ice.

High Pressure Extrusion: This is a frequently used method to disrupt microbial cell. The French pressure cell employs pressures of 10.4×10⁷ Pa (16,000 p.s.i) to break cells open. These apparatus consists of a stainless steel chamber which opens to the outside by means of a needle valve. The cell suspension is placed in the chamber with the needle valve in the closed position. After inverting the chamber, the valve is opened and the piston pushed in to force out any air in the chamber. With the valve in the closed position, the chamber is restored to its original position, placed on a solid based and the required pressure is exerted on the piston by a hydraulic press. When the pressure has been attained the needle valve is opened fractionally to slightly release the pressure and as the cells expand they burst. The valve is kept open while the pressure is maintained so that there is a trickle of ruptured cell which may be collected.

Solid Shear Methods: Mechanical shearing with abrasives may be achieved in Mickle shakers which oscillate suspension vigorously (300-3000 time/min) in the presence of glass beads of 500 nm diameter. This method may result in organelle damage. A more controlled method is to use a Hughes press where a piston forces most cells together with abrasives or deep frozen paste of cells through a 0.25 mm diameter slot in the pressure chamber. Pressures of up to 5.5×10⁷ Pa (8000 p.s.i.) may be used to lyse bacterial preparations.

Liquid Shear Methods: These methods employ blenders, which use high speed reciprocating or rotating blades, homogenizers which use an upward/downward motion of a plunger and ball and microfluidizers or impinging jets which use high velocity passage through small diameter tubes or high velocity impingement of two fluid streams. The blades of blenders are inclined at different angles to permit efficient mixing. Homogenizers are usually operated in short high speed bursts of a few seconds to minimize local heat. These techniques are not generally suitable for microbial cells but even very gentle liquid shear is usually adequate to disrupt animal cells.

Hypotonic/Hypertonic Methods: Cells are exposed to a solution with a much lower (hypotonic) or higher (hypertonic) solute concentration. The difference in solute concentration creates an osmotic pressure gradient. The resulting flow of water into the cell in a hypotonic environment causes the cells to swell and burst. The flow of water out of the cell in a hypertonic environment causes the cells to shrink and subsequently burst.

7. Methods of Concentration and Filtration

Methods of virus particles from host cells include any methods known to those of skill in the art. For example, these methods may include clarification, concentration and diafiltration. One step in the purification process can include clarification of the cell lysate to remove large particulate matter, particularly cellular components, from the cell lysate. Clarification of the lysate can be achieved using a depth filter or by tangential flow filtration. In one embodiment of the present invention, the cell lysate is concentrated. Concentrating the crude cell lysate may include any step known to those of skill in the art. For example, the crude cell lysate may be passed through a depth filter, which consists of a packed column of relatively non-adsorbent material (e.g. polyester resins, sand, diatomaceous earth, colloids, gels and the like). In tangential flow filtration (TFF), the lysate solution flows across a membrane surface which facilitates back diffusion of solute from the membrane surface into the bulk solution. Membranes are generally arranged within various types of filter apparatus including open channel plate and frame, hollow fibers, and tubules.

After clarification and prefiltration of the cell lysate, the resultant heterologous protein supernatant may be concentrated and buffer may be exchanged by diafiltration. The protein supernatant can be concentrated by tangential flow filtration across an ultrafiltration membrane of 10-30K nominal molecular weight cutoff. Ultrafiltration is a pressure modified convective process that uses semi-permeable membranes to separate species by molecular size, shape and/or charge. It separates solvents from solutes of various sizes independent of solute molecular size. Ultrafiltration is gentle, efficient and can be used to simultaneously concentrate and desalt solutions. Ultrafiltration membranes generally have two distinct layers: a thin, dense skin and an open structure of progressively larger voids which are largely open to the permeate side of the ultrafilter. Any species capable of passing through the pores of the skin can therefore freely pass through the membrane. For maximum retention of solute, a membrane is selected that has a nominal molecular weight cut-off well below that of the species being retained. In macromolecular concentration, the membrane enriches the content of the desired biological species and provides filtrate cleared of retained substances. Microsolutes are removed convectively with the solvent. As concentration of the retained solute increases, the ultrafiltration rate diminishes.

In some embodiments of the present invention, an exchange buffer may be used. Buffer exchange, or diafiltration, involving ultrafilters, may be used for the removal and exchange of salts, sugars, non-aqueous solvents or material of low molecular weight.

8. Methods of Virus Purification

Adenovirus purification techniques are well known to those of skill in the art. These adenovirus particles may be purified for example, by employing a number of different purification techniques. Such techniques include those based on sedimentation and chromatography and are described in more detail herein below.

a. Density Gradient Centrifugation

There are two methods of density gradient centrifugation, the rate zonal technique and the isopycnic (equal density) technique, and both can be used when the quantitative separation of all the components of a mixture of particles is required. They are also used for the determination of buoyant densities and for the estimation of sedimentation coefficients.

Particle separation by the rate zonal technique is based upon differences in size or sedimentation rates. The technique involves carefully layering a sample solution on top of a performed liquid density gradient, the highest density of which exceeds that of the densest particles to be separated. The sample is then centrifuged until a desired degree of separation is achieved. For example, centrifugation will take place for a sufficient time to allow for the particles to travel through the gradient to form discrete zones or bands which are spaced according to the relative velocities of the particles. Since the technique is time dependent, centrifugation must be terminated before any of the separated zones pellet at the bottom of the tube. The method has been used for the separation of enzymes, hormones, RNA-DNA hybrids, ribosomal subunits, subcellular organelles, for the analysis of size distribution of samples of polysomes and for lipoprotein fractionations.

The sample is layered on top of a continuous density gradient which spans the whole range of the particle densities which are to be separated. The maximum density of the gradient, therefore, must always exceed the density of the densest particle. During centrifugation, sedimentation of the particles occurs until the buoyant density of the particle and the density of the gradient are equal. At this point no further sedimentation occurs, irrespective of how long centrifugation continues, because the particles are floating on a cushion of material that has a density greater than their own.

Isopycnic centrifugation, in contrast to the rate zonal technique, is an equilibrium method, the particles banding to form zones each at their own characteristic buoyant density. Isopycnic centrifugation depends solely upon the buoyant density of the particle and not its shape or size and is independent of time. Therefore, soluble proteins, which have a very similar density cannot usually be separated by this method, while proteins with dissimilar densities can be effectively separated.

As an alternative to layering the particle mixture to be separated onto a preformed gradient, the sample is initially mixed with the gradient medium to give a solution of uniform density, the gradient “self-forming”, by sedimentation equilibrium, during centrifugation. In this method (referred to as the equilibrium isodensity method), use is generally made of the salts of heavy metals (e.g., caesium or rubidium), sucrose, colloidal silica or Metrizamide.

The sample (e.g., DNA) is mixed homogeneously with, for example, a concentrated solution of caesium chloride. Centrifugation of the concentrated caesium chloride solution results in the sedimentation of the CsCl molecules to form a concentration gradient and hence a density gradient. The sample molecules (DNA), which were initially uniformly distributed throughout the tube now either rise or sediment until they reach a region where the solution density is equal to their own buoyant density, i.e. their isopycnic position, where they will band to form zones. This technique suffers from the disadvantage that often very long centrifugation times (e.g., 36 to 48 hours) are required to establish equilibrium. However, it is commonly used in analytical centrifugation to determine the buoyant density of a particle, the base composition of double stranded DNA and to separate linear from circular forms of DNA.

Many of the separations can be improved by increasing the density differences between the different forms of DNA by the incorporation of heavy isotopes (e.g., N¹⁵) during biosynthesis, a technique used by Leselson and Stahl to elucidate the mechanism of DNA replication in Esherichia coli, or by the binding of heavy metal ions or dyes such as ethidium bromide. Isopycnic gradients have also been used to separate and purify viruses and analyze human plasma lipoproteins.

b. Removing Nucleic Acid Contaminants

The present invention also contemplates nucleases to remove contaminating nucleic acids. Exemplary nucleases include Benzonase®, Pulmozyme®; or any other DNase or RNase commonly used within the art.

Enzymes such as Benzonaze® degrade nucleic acid and have no proteolytic activity. The ability of Benzonase® to rapidly hydrolyze nucleic acids makes the enzyme ideal for reducing cell lysate viscosity. It is well known that nucleic acids may adhere to cell derived particles such as viruses. The adhesion may interfere with separation due to agglomeration, change in size of the particle or change in particle charge, resulting in little if any product being recovered with a given purification scheme. Benzonase® is well suited for reducing the nucleic acid load during purification, thus eliminating the interference and improving yield.

As with all endonucleases, Benzonase® hydrolyzes internal phosphodiester bonds between specific nucleotides. Upon complete digestion, all free nucleic acids present in solution are reduced to oligonucleotides 2 to 4 bases in length.

C. Viral Vectors

A viral vector is a virus that can transfer genetic material from one location to another, such as from the point of application to a target cell of interest. A “viral vector” is meant to include those constructs containing viral sequences sufficient to (a) support packaging of an expression cassette comprising the therapeutic nucleic acid sequences and (b) to ultimately express a recombinant gene construct that has been cloned therein. One of ordinary skill in the art would be familiar with the various types of viruses that are available for use as vectors for gene delivery to a target cell of interest. Each of these is contemplated as a vector in the present invention. Exemplary vectors are discussed below.

1. Adenoviral Vectors

The methods of the present invention may pertain to expression constructs of nucleic acids comprised in adenoviral vectors for delivery to a target cell. Although adenovirus vectors are known to have a low capacity for integration into genomic DNA, this feature is counterbalanced by the high efficiency of gene transfer afforded by these vectors.

Adenoviruses are currently the most commonly used vector for gene transfer in clinical settings. Among the advantages of these viruses is that they are efficient at gene delivery to both nondividing an dividing cells and can be produced in large quantities. In many of the clinical trials for cancer, local intratumor injections have been used to introduce the vectors into sites of disease because current vectors do not have a mechanism for preferential delivery to tumor. In vivo experiments have demonstrated that administration of adenovirus vectors systemically resulted in expression in the oral mucosa (Clayman et al., 1995). Topical application of Ad-βgal and Ad-p53-FLAG on organotypic raft cultures has demonstrated effective gene transduction and deep cell layer penetration through multiple cell layers (Eicher et al., 1996). Therefore, gene transfer strategy using the adenoviral vector is potentially feasible in patients at risk for lesions and malignancies involving genetic alterations in p53.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP (located at 16.8 m.u.), is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

2. Retroviral Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

3. AAV Vectors

Adeno-associated virus (AAV) is an attractive vector system as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte and Carter, 1995).

4. Herpesvirus Vectors

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975). The expression of a genes, the first set of genes to be expressed after infection, is enhanced by the virion protein number 16, or α-transinducing factor (Post et al., 1981; Batterson and Roizman, 1983). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

5. Pox Virus Vectors

Vaccinia virus vectors, which belong to the poxyiral family of viruses, have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxviruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

6. Oncolytic Viral Vectors

Oncolytic viruses are also contemplated as vectors in the present invention. Oncolytic viruses are defined herein to generally refer to viruses that kill tumor or cancer cells more often than they kill normal cells. Exemplary oncolytic viruses include adenoviruses which overexpress ADP. These viruses are discussed in detail in U.S. application Ser. No. 10/810,063, U.S. application Ser. No. 11/076,471, and U.S. application Ser. No. 09/351,778, each of which is specifically incorporated by reference in its entirety into this section of the application and all other sections of the application. Exemplary oncolytic viruses are discussed elsewhere in this specification. One of ordinary skill in the art would be familiar with other oncolytic viruses that can be applied in the pharmaceutical compositions and methods of the present invention.

7. Other Viral Vectors

Other viral vectors also include baculovirus vectors, parvovirus vectors, picornavirus vectors, alphavirus vectors, semiliki forest virus vectors, Sindbis virus vectors, lentivirus vectors, and retroviral vectors. Vectors derived from viruses such as poxvirus may be employed. Additionally, a molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been suggested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

Other viral vectors for application in the compositions and methods of the present invention include those vectors set forth in Tang et al., 2004, which is herein specifically incorporated by reference in its entirety for this section of the application and all other sections of the application.

D. Genetically Modified Viral Vectors

In certain embodiments, the methods of the present invention pertain to viral vectors. Such methods involve the use of a vector construct containing, for example, a heterologous nucleic acid sequence encoding a gene of interest and a means for its expression within a target cell. Alternatively, such a heterologous nucleic acid sequence could be designed to deliver antisense transcripts or interfering RNA sequences when expressed within a target cell. A nucleic acid sequence could simply encode a protein for which large quantities of the protein are desired, or the nucleic acid sequence could be a therapeutic nucleic acid.

1. Nucleic Acids in General

The term “nucleic acid” is well known in the art. A “nucleic acid” as used herein will generally refer to a molecule (i.e., a strand) of DNA, RNA (including RNAi siRNA, and ribozymes), and oligonucleotide, an oligonucleotide comprising CpG site, or a derivative or analog thereof, comprising a nucleobase. The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. The term “heterologous” as used herein will generally refer to a nucleic acid contained within a viral vector which is not naturally found within the genome of the wild type virus from which the viral vector is derived.

These definitions generally refer to a single-stranded molecule, but in specific embodiments will also encompass an additional strand. The additional strand may be partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule As used herein, a single stranded nucleic acid may be denoted by the prefix “ss,” a double stranded nucleic acid by the prefix “ds,” and a triple stranded nucleic acid by the prefix “ts.”

a. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Table 3 shows non-limiting examples of purine and pyrimidine derivatives and analogs. A nucleobase may be comprised in a nucleside or nucleotide, using any chemical or natural synthesis method described herein or known to one of ordinary skill in the art.

TABLE 3
Purine and Pyrmidine Derivatives or Analogs

| Abbr. | Modified base description |
|---|---|
| ac4c | 4-acetylcytidine |
| Chm5u | 5-(carboxyhydroxylmethyl)uridine |
| Cm | 2′-O-methylcytidine |
| Cmnm5s2u | 5-carboxymethylaminomethyl-2-thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| mla | 1-methyladenosine |
| mlf | 1-methylpseudouridine |
| mlg | 1-methylguanosine |
| mlI | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2-methylthiopurine-6-yl)-carbamoyl)threonine |
| Mt6a | N-((9-beta-D-ribofuranosylpurine-6-yl)-N-methyl-carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6-yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

b. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

c. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

d. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference). Any derivative or analog of a nucleoside or nucleotide that is known to those of ordinary skill in the art may be used in the methods and compositions of the present invention. A non-limiting example is a “polyether nucleic acid” and a “peptide nucleic acid.”

2. Therapeutic Nucleic Acids

A “therapeutic nucleic acid” is defined herein to refer to a nucleic acid which can be administered to a subject for the purpose of treating or preventing a disease. The nucleic acid is one which is known or suspected to be of benefit in the treatment of a disease or health-related condition in a subject.

Therapeutic benefit may arise, for example, as a result of alteration of expression of a particular gene or genes by the nucleic acid. Alteration of expression of a particular gene or genes may be inhibition or augmentation of expression of a particular gene. In certain embodiments of the present invention, the therapeutic nucleic acid encodes one or more proteins or polypeptides that can be applied in the treatment or prevention of a disease or health-related condition in a subject. The terms “protein” and “polypeptide” are used interchangeably herein. Both terms refer to an amino acid sequence comprising two or more amino acid residues.

Any nucleic acid known to those of ordinary skill in the art that is known or suspected to be of benefit in the treatment or prevention of a disease or health-related condition is contemplated by the present invention as a therapeutic nucleic acid. The phrase “nucleic acid sequence encoding,” as set forth throughout this application, refers to a nucleic acid which directs the expression of a specific protein or peptide. The nucleic acid sequences include both the DNA strand sequence that is transcribed into RNA and the RNA sequence that is translated into protein. In some embodiments, the nucleic acid includes a therapeutic gene. The term “gene” is used to refer to a nucleic acid sequence that encodes a functional protein, polypeptide, or peptide-encoding unit.

As will be understood by those in the art, the term “therapeutic nucleic acid” includes genomic sequences, cDNA sequences, and smaller engineered gene segments that express, or may be adapted to express, proteins, polypeptides, domains, peptides, fusion proteins, and mutants. The nucleic acid may comprise a contiguous nucleic acid sequence of about 5 to about 12000 or more nucleotides, nucleosides, or base pairs.

Encompassed within the definition of “therapeutic nucleic acid” is a “biologically functional equivalent” of a therapeutic nucleic acid that has proved to be of benefit in the treatment or prevention of a disease or health-related condition. Accordingly, sequences that have about 70% to about 99% homology to a known nucleic acid are contemplated by the present invention.

a. Nucleic Acids that Encode Tumor Suppressors and Pro-Apoptotic Proteins

In some embodiments, a therapeutic nucleic acid may encode a protein or polypeptide that can be applied in the treatment or prevention of cancer or other hyperproliferative disease. Examples of such proteins include, but are not limited to, Rb, CFTR, p16, p21, p27, p57, p73, C-CAM, APC, CTS-1, zac1, scFV ras, DCC, NF-1, NF-2, WT-1, MEN-1, MEN-II, BRCA1, VHL, MMAC1, FCC, MCC, BRCA2, IL-1, IL-2, IL-3, IL-4, IL-5, IL-6, IL-7, IL-8, IL-9, IL-10, IL-11 IL-12, IL-13, GM-CSF, G-CSF, thymidine kinase, mda7, fus, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TAL1, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zac1, ATM, HIC-1, DPC-4, FHIT, PTEN, ING1, NOEY1, NOEY2, OVCA1, MADR2, 53BP2, IRF-1, Rb, zac1, DBCCR-1, rks-3, COX-1, TFPI, PGS, Dp, E2F, ras, myc, neu, raf, erb, fms, trk, ret, gsp, hst, abl, E1A, p300, VEGF, FGF, thrombospondin, BAI-1, GDAIF, or MCC.

A “tumor suppressor” refers to a polypeptide that, when present in a cell, reduces the tumorigenicity, malignancy, or hyperproliferative phenotype of the cell. The nucleic acid sequences encoding tumor suppressor gene amino acid sequences include both the full length nucleic acid sequence of the tumor suppressor gene, as well as non-full length sequences of any length derived from the full length sequences. It should be further understood that sequences may include degenerate codons of the native sequence or sequences which may be introduced to provide codon preference in a specific host cell.

A nucleic acid encoding a tumor suppressor generally refers to a nucleic acid sequence that reduce the tumorigenicity, malignancy, or hyperproliferative phenotype of the cell. Thus, the absence, mutation, or disruption of normal expression of a tumor suppressor gene in an otherwise healthy cell increases the likelihood of, or results in, the cell attaining a neoplastic state. Conversely, when a functional tumor suppressor gene or protein is present in a cell, its presence suppresses the tumorigenicity, malignancy or hyperproliferative phenotype of the host cell. Examples of tumor suppressors include, but are not limited to, APC, CYLD, HIN-1, KRAS2b, p16, p19, p21, p27, p27mt, p53, p57, p73, PTEN, Rb, Uterglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, scFV, ras, MMAC1, FCC, MCC, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide and FUS1. Other exemplary tumor suppressor genes are described in a database of tumor suppressor genes on the world wide web at cise.ufl.edu/˜yyl/HTML-TSGDB/Homepage.html. This database is herein specifically incorporated by reference into this and all other sections of the present application.

One of the best known tumor suppressor genes is p53. p53 is central to many of the cell's anti-cancer mechanisms. It can induce growth arrest, apoptosis and cell senescence. In normal cells p53 is usually inactive, bound to the protein MDM-2, which prevents its action and promotes its degradation. Active p53 is induced after the effects of various cancer-causing agents such as UV radiation, oncogenes and some DNA-damaging drugs. DNA damage is sensed by ‘checkpoints’ in a cell's cycle, and causes proteins such as ATM, Chk1 and Chk2 to phosphorylate p53 at sites that are close to the MDM2-binding region of the protein. Oncogenes also stimulate p53 activation, mediated by the protein p14ARF. Some oncogenes can also stimulate the transcription of proteins which bind to MDM2 and inhibit its activity. Once activated p53 has many anticancer mechanisms, the best documented being its ability to bind to regions of DNA and activate the transcription of genes important in cell cycle inhibition, apoptosis, genetic stability, and inhibition of angiogenesis (Vogelstein et al., 2000). Studies have linked the p53 and pRB tumour suppressor pathways, via the protein p14ARF, raising the possibility that the pathways may regulate each other (Bates et al., 1998).

A nucleic acid encoding a pro-apoptotic protein encode a protein that induces or sustains apoptosis to an active form. The present invention contemplates inclusion of any nucleic acid encoding a pro-apoptotic protein known to those of ordinary skill in the art. Exemplary pro-apoptotic proteins include CD95, caspase-3, Bax, Bag-1, CRADD, TSSC3, bax, hid, Bak, MKP-7, PERP, bad, bcl-2, MST1, bbc3, Sax, BIK, BID, and mda7. One of ordinary skill in the art would be familiar with pro-apoptotic proteins, including those not specifically set forth herein.

b. Nucleic Acids Encoding Cytokines

The term “cytokine” is a generic term for proteins released by one cell population which act on another cell as intercellular mediators. The nucleic acid sequences may encode the full length nucleic acid sequence of the cytokine, as well as non-full length sequences of any length derived from the full length sequences. It being further understood, as discussed above, that the sequence includes the degenerate codons of the native sequence or sequences which may be introduced to provide codon preference in a specific host cell.

Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factors (FGFs) such as FGF-α and FGF-β; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-α; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-11, IL-12; IL-13, IL-14, IL-15, IL-16, IL-17, IL-18, LIF, G-CSF, GM-CSF, M-CSF, EPO, kit-ligand or FLT-3.

A non limiting example of growth factor cytokines involved in wound healing include: epidermal growth factor, platelet-derived growth factor, keratinocyte growth factor, hepatycyte growth factor, transforming growth factors (TGFs) such as TGF-α and TGF-β, and vascular endothelial growth factor (VEGF). These growth factors trigger mitogenic, motogenic and survival pathways utilizing Ras, MAPK, PI-3K/Akt, PLC-gamma and Rho/Rac/actin signaling. Hypoxia activates pro-angiogenic genes (e.g., VEGF, angiopoietins) via HIF, while serum response factor (SRF) is critical for VEGF-induced angiogenesis, re-epithelialization and muscle restoration. EGF, its receptor, HGF and Cox2 are important for epithelial cell proliferation, migration re-epithelialization and reconstruction of gastric glands. VEGF, angiopoietins, nitric oxide, endothelin and metalloproteinases are important for angiogenesis, vascular remodeling and mucosal regeneration within ulcers. (Tarnawski, 2005)

Another example of a cytokine is IL-10. IL-10 is a pleiotropic homodimeric cytokine produced by immune system cells, as well as some tumor cells (Ekmekcioglu et al., 1999). Its immunosuppressive function includes potent inhibition of proinflammatory cytokine synthesis, including that of IFNγ, TNFα, and IL-6 (De Waal et al., 1991). The family of IL-10-like cytokines is encoded in a small 195 kb gene cluster on chromosome 1q32, and consists of a number of cellular proteins (IL-10, IL-19, IL-20, MDA-7) with structural and sequence homology to IL-10 (Kotenko et al., 2000; Gallagher et al., 2000; Blumberg et al., 2001; Dumoutier et al., 2000; Knapp et al., 2000; Jiang et al., 1995; Jiang et al., 1996).

Still another example of a cytokine is MDA-7. This cytokine has been characterized as an IL-10 family member and is also known as IL-24. Chromosomal location, transcriptional regulation, murine and rat homologue expression, and putative protein structure all allude to MDA-7 being a cytokine (Knapp et al., 2000; Schaefer et al., 2000; Soo et al., 1999; Zhang et al., 2000). Similar to GM-CSF, TNFα, and IFNγ transcripts, all of which contain AU-rich elements in their 3'UTR targeting mRNA for rapid degradation, MDA-7 has three AREs in its 3′UTR¹⁷. Mda-7 mRNA has been identified in human PBMC (Ekmekcioglu, et al., 2001), and although no cytokine function of human MDA-7 protein has been previously reported, MDA-7 has been designated as IL-24 based on the gene and protein sequence characteristics (NCBI database accession XM_{—}001405).

c. Nucleic Acids Encoding Enzymes

Other examples of therapeutic nucleic acids include nucleic acids encoding enzymes. Examples include, but are not limited to, ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyme, a pectinesterase, a peroxidase, a phosphatase, a phospholipase, a phosphorylase, a polygalacturonase, a proteinase, a peptidase, a pullanase, a recombinase, a reverse transcriptase, a topoisomerase, a xylanase, a reporter gene, an interleukin, or a cytokine.

Further examples of therapeutic genes include the gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, low-density-lipoprotein receptor, porphobilinogen deaminase, factor VIII, factor IX, cystathione beta.-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase, galactose-1-phosphate uridyltransferase, phenylalanine hydroxylase, glucocerebrosidase, sphingomyelinase, α-L-iduronidase, glucose-6-phosphate dehydrogenase, glucosyltransferase, HSV thymidine kinase, or human thymidine kinase.

d. Nucleic Acids Encoding Hormones

Therapeutic nucleic acids also include nucleic acids encoding hormones. Examples include, but are not limited to, growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, and thyrotropin releasing hormone.

e. Nucleic Acids Encoding Antigens

A therapeutic gene may encode antigens present in tumors, pathogens, or immune effectors involved in autoimmunity. These genes may be applicable in vaccine or immune therapy of neoplasias, infectious diseases and autoimmune diseases.

Tumor Antigens. In certain embodiments, a therapeutic nucleic acid may encode a tumor antigen. Tumor antigens are well-known to those of ordinary skill in the art. Examples include, but are not limited to, those described by Dalgleish, (2004), Finn, (2003), and Hellstrom and Hellstrom, (2003), each of which is herein incorporated by reference in its entirety. Other examples can be found on the world wide web at bioinfo.org.cn/hptaa/search.php, which is herein specifically incorporated by reference.

Microorganism Antigens. In certain embodiments, a therapeutic nucleic acid may encode a microorganism antigen. The term “microorganism” includes viruses, bacteria, microscopic fungi, protozoa and other microscopic parasites. A “microorganism antigen” refers to a polypeptide that, when presented on the cell surface by antigen presenting cells (APCs), induces an immune response. This response may include a cytotoxic T cell response or the production of antibodies or both.

Examples of viruses from which microorganism antigens may be derived include: human herpes viruses (HHVs)-1 through 8; herpes B virus; HPV-16, 18, 31, 33, and 45; hepatitis viruses A, B, C, δ; poliovirus; rotavirus; influenza; lentiviruses; HTLV-1; HTLV-2; equine infectious anemia virus; eastern equine encephalitis virus; western equine encephalitis virus; venezuelan equine encephalitis virus; rift valley fever virus; West Nile virus; yellow fever virus; Crimean-Congo hemorrhagic fever virus; dengue virus; SARS coronavirus; small pox virus; monkey pox virus and/or the like.

Examples of viral microorganisms include, but are not limited to: retroviridae, flaviridae, coronaviridae, picornaviridae, togaviridae, rhabdoviridae, paramyxoviridae, orthomyxoviridae, bunyaviridae, arenaviridae, reoviridae, polyomaviridae, papillomaviridae, herpesviridae and hepadnaviridae.

Examples of retroviridae include lentiviruses such as HIV-1, HIV-2, SIV, FIV, Visna, CAEV, BIV and EIAV. Genes encoded by lentiviruses may include gag, pol, env, vif, vpr, vpu, nef, tat, vpx and rev. Other examples of retroviruses include alpha retroviruses such as avian leukosis virus, avian myeloblastosis virus, avian sarcoma virus, fujinami sarcoma virus and rous sarcoma virus. Genes encoded by alpha retroviruses may include gag, pol and env. Further examples of retroviruses include beta retroviruses such as jaagsiekte sheep retrovirus, langur virus, Mason-Pfizer monkey virus, mouse mammary tumor virus, simian retrovirus 1 and simian retrovirus 2. Genes encoded by beta retroviruses may include gag, pol, pro and env. Still further examples of retroviruses include delta retroviruses such as HTLV-1, HTLV-2, bovine leukemia virus, and baboon T cell leukemia virus. Genes encoded by delta retroviruses may include gag, pol, env, tax and rex. Still further examples of retrovirus include spumaviruses such as bovine, feline, equine, simian and human foamy viruses. Genes encoded by spumaviruses may include gag, pol, env, bel-1, bel-2 and bet.

Examples of flaviridae include but are not limited to: hepatitis C virus, mosquito borne yellow fever virus, dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, louping III virus, Powassan virus, Omsk hemorrhagic fever virus, the genus rubivirus (rubella virus) and the genus pestivirus (mucosal disease virus, hog cholera virus, border disease virus). Genes encoded by flaviviruses include the flavivirus polyprotein from which all flavivirus proteins are derived. Nucleic acid sequences encoding the flavivirus polyprotein may include sequences encoding the final processed flavivirus protein products such as C, prM, E, NS1, NS2A, NS2B, NS3, NS4A, NS4B and NS5.

Examples of coronaviridae include but are not limited to: human respiratory coronaviruses such as SARS and bovine coronaviruses. Genes encoded by coronaviridae may include pol, S, E, M and N.

Examples of picornaviridae include but are not limited to the genus Enterovirus (poliovirus, Coxsackie virus A and B, enteric cytopathic human orphan (ECHO) viruses, hepatitis A virus, simian enteroviruses, murine encephalomyelitis (ME) viruses, poliovirus muris, bovine enteroviruses, porcine enteroviruses, the genus cardiovirus (encephalomyocarditis virus (EMC), mengovirus), the genus rhinovirus (human rhinoviruses including at least 113 subtypes; other rhinoviruses) and the genus apthovirus (foot and mouth disease (FMDV). Genes encoded by picornaviridae may include the picornavirus polyprotein. Nucleic acid sequences encoding the picornavirus polyprotein may include sequences encoding the final processed picornavirus protein products such as VPg, VPO, VP3, VP1, 2A, 2B, 2C, 3A, 3B, 3C and 3D.

Examples of togaviridae include but are not limited to including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis Eastern equine encephalitis virus). Examples of genes encoded by togaviridae may include genes coding for nsP1, nsP2, nsP3 nsP4, C, E1 and E2.

Examples of rhabdoviridae include, but are not limited to: including the genus vesiculovirus (VSV), chandipura virus, Flanders-Hart Park virus) and the genus lyssavirus (rabies virus). Examples of genes encoded by rhabdoviridae may include N, P, M, G, and L.

Examples of filoviridae include Ebola viruses and Marburg virus. Examples of genes encoded by filoviruses may include NP, VP35, VP40, GP, VP35, VP24 and L.

Examples of paramyxoviruses include, but are not limited to: including the genus paramyxovirus (parainfluenza virus type 1, sendai virus, hemadsorption virus, parainfluenza viruses types 2 to 5, Newcastle disease Virus, mumps virus), the genus morbillivirus (measles virus, subacute sclerosing panencephalitis virus, distemper virus, Rinderpest virus), the genus pneumovirus (respiratory syncytial virus (RSV), bovine respiratory syncytial virus and pneumonia virus of mice). the family paramyxoviridae, including the genus Paramyxovirus (Parainfluenza virus type 1, Sendai virus, hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice). Examples of genes encoded by paramyxoviridae may include N, P/C/V, P/C/V/R, M, F, HN, L, V/P, NS1, NS2, SH and M2.

Examples of orthomyxoviridae include influenza viruses. Examples of genes encoded by orthomyxoviridae may include PB1, PB2, PA, HA, NP, NA, M1, M2, NS1 and NS2.

Examples of bunyaviruses include, but are not limited to: the genus bunyvirus (bunyamwera and related viruses, California encephalitis group viruses), the genus phlebovirus (sandfly fever Sicilian virus, Rift Valley fever virus), the genus nairovirus (Crimean-Congo hemorrhagic fever virus, Nairobi sheep disease virus) and the genus uukuvirus (uukuniemi and related viruses). Examples of genes encoded by bunyaviruses may include N, G1, G2 and L.

Examples of arenaviruses include, but are not limited to: lymphocytic choriomeningitis virus, lassa fever virus, Argentine hemorrhagic fever virus, Bolivian hemorrhagic fever virus and Venezuelan hemorrhagic fever virus. Examples of genes encoded by arenaviruses may include NP, GPC, L and Z.

Examples of reoviruses include, but are not limited to: the genus orthoreovirus (multiple serotypes of both mammalian and avian retroviruses), the genus orbivirus (Bluetongue virus, Eugenangee virus, Kemerovo virus, African horse sickness virus, and Colorado Tick Fever virus) and the genus rotavirus (human rotavirus, Nebraska calf diarrhea virus, murine rotavirus, simian rotavirus, bovine or ovine rotavirus, avian rotavirus). Examples of genes encoded by reoviruses may include genome segments named for their corresponding protein products, such as VP1, VP2, VP3, VP4, NSP1, NSP3, NSP2, VP7, NSP4, NSP5 and NSP6.

Examples of polyomaviridae include, but are not limited to BK and JC viruses. Examples of genes encoded by polyomaviruses may include Agno, P2, VP3, VP2, VP1, large T and small t.

Examples of papillomaviridae include, but are not limited to: HPV-16 and HPV-18. Examples of genes encoded by papillomaviruses may include E1, E2, E3, E4, E5, E6, E7, E8, L1 and L2.

Examples of herpesviridae include, but are not limited to: Human Herpes Virus (HHV) 1, HHV2, HHV3, HHV4, HHV5, HHV6, HHV7 and HHV8. Examples of genes encoded by herpesviruses may include γ₁34.5, ORF P, ORFO, αO, U_{L}1 through U_{L}56, α4, α22, U_{S}2 through U_{S}12, Ori_{S}TU and LATU.

Examples of hepadnaviruses include but is not limited to hepatitis B virus. Examples of genes encoded by hepadnaviruses may include S, C, P and X.

Examples of fungi from which microorganism antigens may be derived include: histoplasma capsulatum; aspergillus; actinomyces; candida, streptomyces and/or the like.

Examples of protozoa or other microorganisms from which antigens may be derived include plasmodium falciparum, plasmodium vivax, plasmodium ovale, plasmodium malariae, and the like. Genes derived from plasmodium species may include PyCSP, MSP1, MSP4/5, Pvs25 and Pvs28.

Examples of bacteria from which microorganism antigens may be derived include: mycobacterium tuberculosis; yersinia pestis; rickettsia prowazekii; rickettsia rickettsii; francisella tularensis; bacillus anthracis; helicobacter pylori; salmonella typhi; borrelia burgdorferi; streptococcus mutans; and/or the like. Genes derived from mycobacterium tuberculosis may include 85A, 85B, 85C and ESAT-6. Genes derived from yersinia pestis may include lcrV and caf1. Genes derived from rickettsia species may include ospA, invA, ompA, ompB, virB, cap, tlyA and tlyC. Genes derived from francisella tularensis may include nucleoside diphosphate kinase, isocitrate dehydrogenase, Hfq and ClpB. Genes derived from bacillus anthracis may include PA, BclA and LF. Genes derived from helicobacter pylori may include hpaA, UreB, hspA, hspB, hsp60, VacA, and cagE. Genes derived from salmonella typhi may include mpC, aroC, aroD, htrA and CS6. Genes derived from borrelia burgdorferi may include OspC.

Examples of fungi from which microorganism antigens may be derived include: histoplasma; ciccidis; immitis; aspargillus; actinomyces; blastomyces; candida, streptomyces and/or the like.

Examples of protozoa or other microorganisms from which antigens may be derived include: plasmodium falciparum; plasmodium vivax; plasmodium ovale; plasmodium malariae; giadaria intestinalis and/or the like.

The microorganism antigen may be a glucosyltransferases derived from Streptococci mutans. The glucosyltransferases mediate the accumulation of S. mutans on the surface of teeth. Inactivation of glucosyltransferase has been demonstrated to cause a reduction in dental caries (Devulapalle and Mooser, 2001).

Another example an antigen derived from Streptococci mutans is PAc protein. PAc is a 190-kDa surface protein antigen involved in the colonization of Streptococci mutans, which mediates the initial adherence of this organism to tooth surfaces. Recently, it has been reported that in vivo administration of plasmid DNA encoding a fusion protein of amino acid residues 1185-1475 encoded by the glucosyltransferase-B of S. mutans, and amino acid residues 222-965 encoded by the PAc gene of S. mutans elicited an immune response against these respective gene products (Guo et al., 2004).

f. Nucleic Acids Encoding Antibodies

In certain embodiments, a therapeutic nucleic acid may encode an antibody. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. In certain embodiments, the antibody is a single chain antibody. Single-chain antibodies are described in U.S. Pat. No. 4,946,778 and U.S. Pat. No. 5,888,773, each of which are hereby incorporated by reference.

g. Ribozymes

In certain embodiments, a therapeutic nucleic acid may be a ribozyme. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

h. RNAi

In certain embodiments of the present invention, the therapeutic nucleic acid may be an RNAi. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

The endoribonuclease Dicer is known to produce two types of small regulatory RNAs that regulate gene expression: small interfering RNAs (siRNAs) and microRNAs (miRNAs) (Bernstein et al., 2001; Grishok et al., 2001; Hutvagner et al., 2001; Ketting et al., 2001; Knight and Bass, 2001). In animals, siRNAs direct target mRNA cleavage (Elbashir et al., 2001), whereas miRNAs block target mRNA translation (Reinhart et al., 2000; Brennecke et al., 2003; Xu et al., 2003). Recent data suggest that both siRNAs and miRNAs incorporate into similar perhaps even identical protein complexes, and that a critical determinant of mRNA destruction versus translation regulation is the degree of sequence complementary between the small RNA and its mRNA target (Hutvagner and Zamore, 2002; Mourelatos et al., 2002; Zeng et al., 2002; Doench et al., 2003; Saxena et al., 2003).

i. Antisense Constructs

In certain embodiments of the present invention, the therapeutic nucleic acid may be an antisense construct. Antisense methodology takes advantage of the fact that nucleic acids tend to pair with “complementary” sequences.” By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

3. Control Regions

In order for a viral vector to effect expression of a transcript encoding a therapeutic gene, the polynucleotide encoding the therapeutic gene will be under the transcriptional control of a promoter and a polyadenylation signal. Such a linkage between an exogenous nucleic acid and its corresponding control regions may be defined herein as an “expression cassette”. A “promoter” refers to a DNA sequence recognized by the synthetic machinery of the host cell, or introduced synthetic machinery, that is required to initiate the specific transcription of a gene. A polyadenylation signal refers to a DNA sequence recognized by the synthetic machinery of the host cell, or introduced synthetic machinery, that is required to direct the addition of a series of nucleotides on the end of the mRNA transcript for proper processing and trafficking of the transcript out of the nucleus into the cytoplasm for translation. The phrases “operatively linked,” “under control,” and “under transcriptional control” mean that the promoter is in the correct location in relation to the polynucleotide to control RNA polymerase initiation and expression of the polynucleotide.

a. Promoters and Enhancers

In order for the expression cassette to effect expression of a transcript, the nucleic acid encoding gene will be under the transcriptional control of a promoter. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Any promoter known to those of ordinary skill in the art that would be active in a cell in any cell in a subject is contemplated as a promoter that can be applied with the methods of the present invention. In certain embodiments, for example, the promoter is a constitutive promoter, an inducible promoter, or a repressible promoter. The promoter can also be a tissue selective promoter. A tissue selective promoter is defined herein to refer to any promoter which is relatively more active in certain tissue types compared to other tissue types. Thus, for example, a liver-specific promoter would be a promoter which is more active in liver compared to other tissues in the body. One type of tissue-selective promoter is a tumor selective promoter. A tumor selective promoter is defined herein to refer to a promoter which is more active in tumor tissue compared to other tissue types. There may be some function in other tissue types, but the promoter is relatively more active in tumor tissue compared to other tissue types. Examples of tumor selective promoters include the hTERT promoter, the CEA promoter, the PSA promoter, the probasin promoter, the ARR2PB promoter, and the AFP promoter.

The promoter will be one which is active in the target cell. For instance, where the target cell is a keratinocyte, the promoter will be one which has activity in a keratinocyte. Similarly, where the cell is an epithelial cell, skin cell, mucosal cell or any other cell that can undergo transformation by a papillomavirus, the promoter used in the embodiment will be one which has activity in that particular cell type.

A promoter may be one naturally associated with a gene or sequence, as may be obtained by isolating the 5′-non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Furthermore, it is contemplated the control sequences that direct transcription and/or expression of sequences within non-nuclear organelles such as mitochondria, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. 2001, incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The particular promoter that is employed to control the expression of the nucleic acid of interest is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter and the Rous sarcoma virus long terminal repeat can be used. The use of other viral or mammalian cellular or bacterial phage promoters which are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter which is active in specific cells, such as tyrosine (melanoma), alpha-fetoprotein and albumin (liver tumors), CC10 (lung tumors) and prostate-specific antigen (prostate tumor) will permit tissue-specific expression of the therapeutic nucleic acids set forth herein. The following is a non-limited list of promoter/elements which may be employed in the context of the present invention: Immunoglobulin Heavy Chain, Immunoglobulin Light Chain, T-Cell Receptor, HLA DQ a and/or DQ β, β-Interferon, Interleukin-2, Interleukin-2 Receptor, MHC Class II 5, MHC Class II HLA-DRa, β-Actin, Muscle Creatine Kinase (MCK), Prealbumin (Transthyretin), Elastase I, Metallothionein (MTII), Collagenase, Albumin, α-Fetoprotein, t-Globin, β-Globin, c-fos, c-HA-ras, Insulin promoter, Neural Cell Adhesion Molecule (NCAM) promoter, α₁-Antitrypsin promoter, H2B (TH2B) Histone promoter, Mouse and/or Type I Collagen promoter, Glucose-Regulated Proteins (GRP94 and GRP78), Rat Growth Hormone, Human Serum Amyloid A (SAA), Troponin I (TN I), Platelet-Derived Growth Factor promoter, Polyomavirus promoters, Retrovirus promoters, Papilloma Virus promoters, Hepatitis B Virus promoters and Cytomegalovirus (CMV) promoters. This list is not intended to be exhaustive of all the possible promoter and enhancer elements, but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have very similar modular organization.

Additionally, any promoter/enhancer combination (as per the Eukaryotic Promoter Data Base EPDB) could also be used to drive expression of a gene. Use of a T3, T7, or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional expression vector.

Further selection of a promoter that is regulated in response to specific physiologic signals can permit inducible expression of a construct. For example, with the polynucleotide under the control of the human PAI-1 promoter, expression is inducible by tumor necrosis factor.

b. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

c. IRES

In certain instances, internal ribosome entry sites (IRES) elements may be incorporated into a nucleic acid to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each of which is herein incorporated by reference). One of ordinary skill in the art would be familiar with the application of IRES in gene therapy.

d. Multiple Cloning Sites

Nucleic acids can include a multiple cloning site (MCS), which is a region that contains multiple restriction enzyme sites, any of which can be used in conjunction with standard recombinant technology to digest the vector. See Carbonelli et al., (1999); Levenson et al., (1998); Cocea, (1997). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997).

e. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Development of Size Exclusion Chromatography/High Performance Liquid Chromatography Assay

Currently, adenovirus product purity is measured by an ion exchange HPLC method (IEX-HPLC) using the Source 15Q resin packed in a 1 mL Resource Q column (TR057 Waters HPLC for QC Adenoviral Samples). Since adenovirus product is purified using the same Source 15Q resin, further analysis using the same chemistry on a HPLC is not likely to detect all impurities that are not removed during the purification process. Ion Exchange Chromatography-HPLC separation is based on differences in molecule charge. Thus, an orthogonal size exclusion-HPLC method (SEC-HPLC), which is based on differences in molecule size for separation, is expected to better detect the presence of residual impurities in the adenovirus product and provide additional purity information.

### Materials and Methods

SEC-HPLC Conditions

A) Column

Bio-Sep-SEC-S3000, Phenomenex, P/N 00H-2146-K0, S/N 319165-13

B) Mobile Running Buffer

0.1 M Na₂HPO₄, pH 7.00

C) Elution

Isocratic elution for 30 minutes at a flow rate of 1 ml/minute

D) Detection

Photodiode array detector at 215 nm

E) Sample Injection Volume

200 μl

F) HPLC System

Beckman system Gold with a photodiode array detector (PD)

A Bio-Sep-SEC-S3000 (Phenomenex, P/N 00H-2146-K0, S/N 319165-13) size exclusion column (column volume=10 mL) was selected based on its separation of a wide range of molecular weights. The column was equilibrated with a phosphate buffer at a neutral pH (pH=7.00). After column equilibration, a 200 μl diluted purified adenovirus sample was injected onto the column. The sample was eluted out from the column using an isocratic elution at a flow rate of 1 mL/min. The 215 nm UV wavelength was selected for improved detection sensitivity. The length of the isocratic elution was 30 minutes. At the end of the 30 minute elution, the column was ready to be used for analysis of a new sample.

### Results

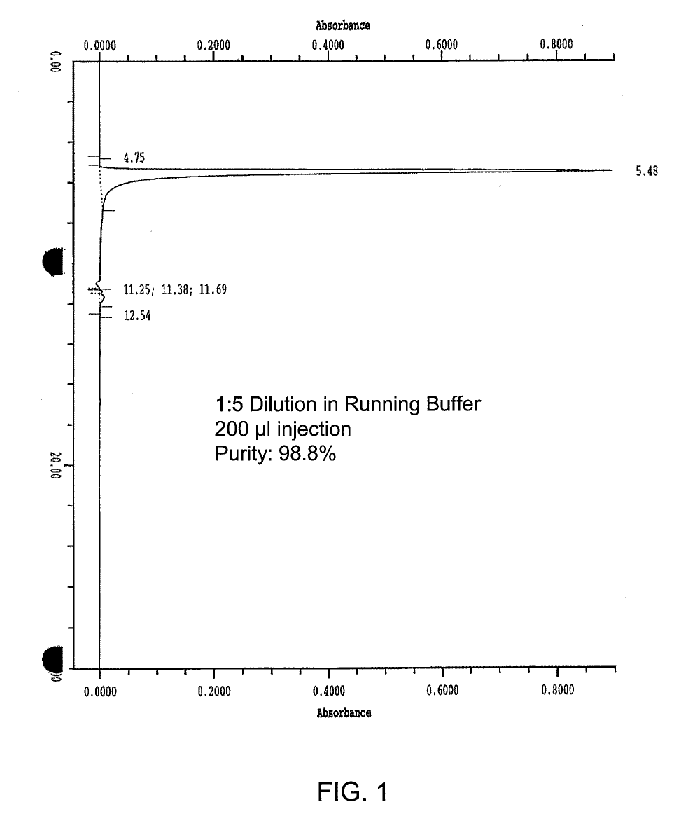

Analysis of the adenovirus sample material (QC reference material B2949801) demonstrated a main peak at 5.48 minutes of retention time (RT), as well as two small peaks at an RT of 11.25 and 12.54 minutes. The main peak RT is believed to be the intact adenovirus particles, while the small peaks are thought to be small molecular weight impurities present in the adenovirus product. FIG. 1 shows the representative SEC-HPLC chromatogram of the adenovirus sample.

### Example 2

### Analysis of the Size Exclusion Chromatography-HPLC Peaks

### Materials and Methods

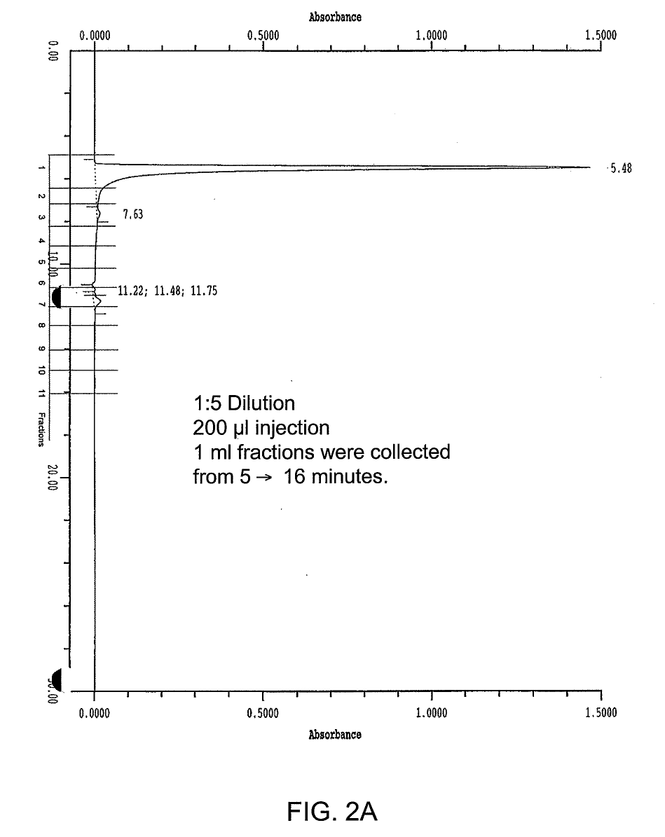

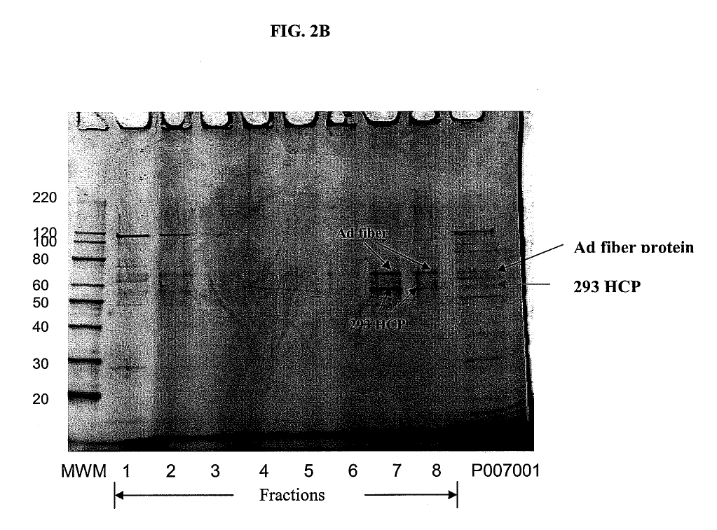

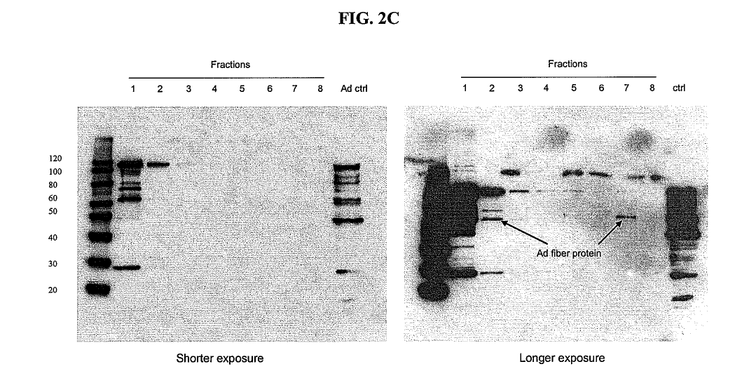

Using the materials and methods described in Example 1 for SEC-HPLC, a wave reactor produced adenovirus sample (adenovirus lot #P007001) was diluted 5-fold with the SEC-HPLC running buffer. Subsequently, 200 μl of this diluted sample was loaded onto the SEC-HPLC column. One milliliter fractions were collected from the HPLC column during the elution step. The SEC-HPLC profile and fraction collection scheme is shown in FIG. 2A. After elution, the fractions (20 μl per well) were analyzed by SDS-PAGE using Sypro-Orange staining, FIG. 2B. Subsequently, these fractions were analyzed by Western blot using a polyclonal antibody reactive to adenovirus serotype 5.

### Results

Two unique protein bands were observed in fractions 7 and 8. Based on molecular weight alignment, the 2 bands were tentatively identified as the adenovirus fiber protein (the top band) and a 293 host cell protein (the lower band). Fraction 1 appeared be intact adenovirus particles, which was confirmed by anti-adenovirus Western blot analysis, FIG. 2C. The Western blot result confirmed that the first peak (fraction 1) is intact adenovirus particles. Under longer exposure, the top protein band identified in fractions 7 and 8 was identified to be the fiber protein of adenovirus. The lower band did not react with the anti-Ad5 antibody, suggesting it is not an adenovirus component protein and likely be a 293 host cell protein.

### Example 3

### SEC-HPLC Analysis of Purified Adenovirus Products

### Materials and Methods

Using the materials and methods described in Example 1, the following four lots of adenovirus products were analyzed by the SEC-HPLC method as shown in Table 4.

TABLE 4

| Construct | Lot Number | Production Process |
|---|---|---|
| INGN 007 | P007001 | Wave Suspension Cells |
| INGN 241 | P241001 | Wave Suspension Cells |
| INGN 241 | B2119901 | Cell Cube Adherent Cells |
| INGN 201 | B2949801 | Cell Cube Adherent Cells |

Additionally, these same lots were analyzed by IEX-HPLC for comparison purposes as described in Experiment 1. The peak areas for both SEC-HPLC and the comparative IEX-HPLC were auto-integrated by the HPLC system and used to calculate the adenovirus product purity.

### Results

Since the adenovirus products were purified using the same resin (Source 15Q) as was used for the IEX-HPLC (TR057 Waters HPLC for QC Adenoviral Samples) analysis, analysis of the adenovirus product using an orthogonal SEC-HPLC resulted in the detection of residual impurities which could not be determined by the comparative IEX-HPLC. Table 4. Thus, this data appears to suggest that the SEC-HPLC method is a more informative method for adenovirus product purity determination compared to the current IEX-HPLC method.

TABLE 5

Purity (%)

| Adenovirus product | SEC-HPLC | IEX-HPLC (TR057) |
|---|---|---|
| B2949801 | 98.8 | ≧99.7 |
| B2119901 | 99.0 | ≧99.8 |
| P241001 | 98.4 | 100 |
| P007001 | 97.5 | 100 |

### Example 4

### SEC-HPLC for Adenovirus Quantitation

### Materials and Methods

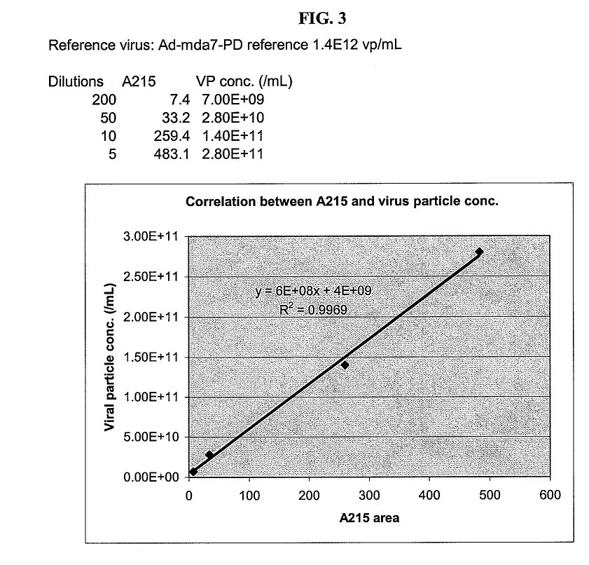

Using the materials and methods of Experiment 1, the SEC-HPLC method was used to quantify the level of adenoviral protein in a test sample in reference to a standard curve. Accordingly, the PD reference adenovirus (Ad-mda7) was used to generate a standard curve correlating 215 nm absorbance (quantified as the 215 nm peak area by the HPLC system) to adenovirus particle concentration. The standard curve is shown in FIG. 3.

### Results

There was a high correlation coefficient (R²=0.9969) between the 215 nm absorbance and the injected virus particle concentration. Thus, the results indicate that the SEC-HPLC method can be used to measure the viral particle concentration of a purified adenovirus sample.

### Example 5

### SEC-HPLC as a Stability Indicating Assay for Adenovirus Storage

Loss of adenovirus stability in storage is generally caused by changes in the virus surface structural proteins, such as loss of the fiber and the penton base. Denatured adenovirus particles have a smaller size than that of the intact virus particles and thus, SEC-HPLC is expected to be able to detect the changes and may therefore serve as a stability indicating assay.

### Materials and Methods

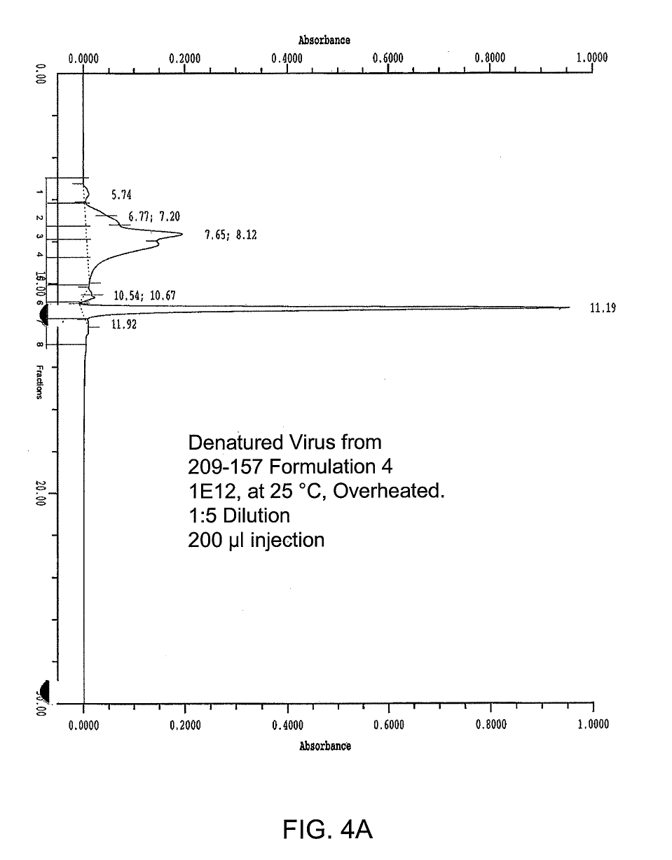

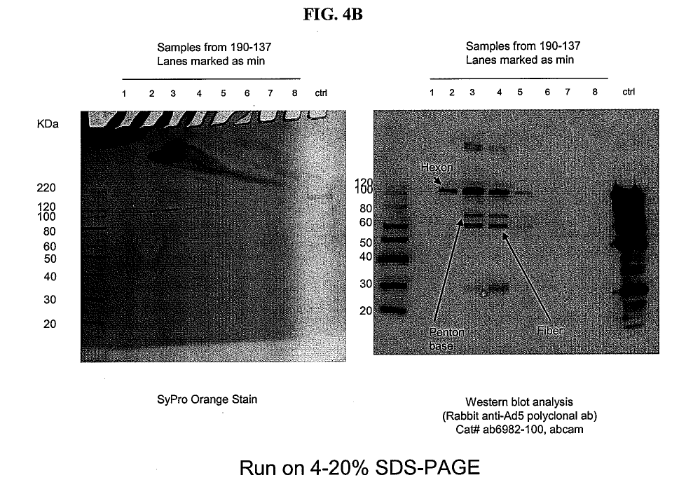

Using the materials and methods of Example 1, a denatured adenovirus product was analyzed by SEC-HPLC assay. The denatured adenovirus was mixed and diluted 5-fold using the SEC-HPLC running buffer. 200 μl of the diluted virus was injected onto the SEC-HPLC column. Fractions in 1 milliliter volumes were collected between 5 and 12 minutes of retention time. The fractions were analyzed by SDS-PAGE and anti-Ad5 western blot. The HPLC chromatogram is shown in FIG. 4A. Fraction markings were added manually for reference purpose (not to scale).

### Results

The HPLC chromatogram is significantly different from that of an intact adenovirus sample (FIG. 4A). The peak at RT of 5.48 minute, representing intact adenovirus particles, was no longer detectable in the denatured sample. Instead, a broad peak between RT of 6.77 to 8.12 showed up in the chromatogram. The large peak at RT 11.19 minute was shown in other studies to be caused by excipients (PEG-3000 and Tween-80) used in the formulation study.

As expected fractions 2, 3, 4, and 5, which make up the broad peak, contained adenovirus structural proteins. FIG. 4B. It is interesting to note that fraction 2 contained only Hexon protein and with no detectable Penton base and Fiber proteins, suggesting this fraction was made up mainly of the large Group of Nine Hexons. The smaller peripentonal Hexons, Penton base, Fiber and core proteins showed up in the following on fractions. The data appear to suggest that the icosahedral structure of an intact adenovirus particle was dissociated into individual structural components. The destruction of the icosahedral structure may ultimately lead to the precipitation generally associated with a denatured adenovirus product. No protein was detected in fraction 7 which covers the main peak at RT 11.19 minute. This is consistent with the discussion above that this peak was caused by the excipients (PEG-3000 and Tween-80) used in the formulation buffer.

A SEC-HPLC method was developed for analysis of purified adenovirus product. Relative to the current IEX-HPLC method used for adenovirus product purity measurement, the SEC-HPLC appears to provide more informative result on product purity and was able to detect impurities that were not detected by the IEX-HPLC. The SEC-HPLC method can also be used to quantify the virus particle concentration of a purified adenovirus sample. Furthermore, the SEC-HPLC method was shown to be able to detect changes in the structural proteins of a denatured adenovirus sample. The result appeared to suggest that the icosahedral structure of an intact adenovirus particle was dissociated into smaller viral components during the course of virus inactivation. Therefore, the SEC-HPLC assay can be a stability indicating assay for adenovirus product.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
PCT App. No. WO 96/27677
PCT App. No. WO 97/08298
PCT App. No. WO 98/00524
PCT App. No. WO 98/22588
U.S. patent application Ser. No. 09/351,778
U.S. patent application Ser. No. 10/810,063
U.S. patent application Ser. No. 11/076,471
U.S. Pat. No. 3,917,527
U.S. Pat. No. 4,000,098
U.S. Pat. No. 4,332,717
U.S. Pat. No. 4,352,883
U.S. Pat. No. 4,683,202
U.S. Pat. No. 4,743,680
U.S. Pat. No. 4,771,128
U.S. Pat. No. 4,797,368
U.S. Pat. No. 4,894,439
U.S. Pat. No. 4,908,434
U.S. Pat. No. 4,920,196
U.S. Pat. No. 4,946,778
U.S. Pat. No. 5,139,941
U.S. Pat. No. 5,252,216
U.S. Pat. No. 5,354,855
U.S. Pat. No. 5,447,859
U.S. Pat. No. 5,491,096
U.S. Pat. No. 5,672,344
U.S. Pat. No. 5,837,520
U.S. Pat. No. 5,888,773
U.S. Pat. No. 5,902,722
U.S. Pat. No. 5,925,565
U.S. Pat. No. 5,928,906
U.S. Pat. No. 5,935,819
U.S. Pat. No. 6,194,191
U.S. Pat. No. 6,261,823
U.S. Pat. No. 6,316,185
U.S. Pat. No. 6,630,299
Albrechtsen et al., J. Virol. Methods, 28:245-256, 1990.
Bates et al., Nature, 395:124-125, 1998.
Batterson and Roizman, J. Virol., 46:371-377, 1983.
Berg et al., BioTechniques, 14:972-978, 1993.
Bernstein et al., Nature, 409:363-366, 2001.
Bett, Proc. Natl. Acad. Sci. USA, 91:8802-8806, 1994.
Blumberg et al., Cell, 104:9-19, 2001.
Bosher and Labouesse, Nature Cell Biol., 2:E31-E36, 2000.
Brennecke et al., Cell, 113:25-36, 2003.
Caley et al., J. Virology, 71:3031-3038, 1997.
Carbonelli et al., FEMS Microbiol. Lett., 177:75-82, 1999.
Chandler et al., Proc. Natl. Acad. Sci. USA, 94:3596-601, 1997.
Chang et al., Hepatology, 14:134A, 1991.
Clark et al., Hum. Gene Ther., 6:1329-1341, 1995.
Clayman et al., Cancer Res., 55:1-6, 1995.
Cocea, Biotechniques, 23:814-816, 1997.
Coffin, In: Virology, Fields et al. (Eds.), Raven Press, NY, 1437-1500, 1990.
Cook et al., Cell, 27:487-496, 1981.
Couch et al., Am. Rev. Resp. Dis., 88:394-403, 1963.
Cristiano et al., Cancer Detect. Prev., 22:445-454, 1998.
Crooks et al., J. Chromatog., 502:59-68, 1990.
Dalgleish, Expert Rev. Vaccines, 3:665-668, 2004.
Davis et al., Curr. Biol., 6:146-148, 1996.
de Waal et al., J. Exp. Med., 174:1209-1220, 1991.
DeLuca et al., J. Virol., 56:558-570, 1985.
Devulapalle and Mooser, J. Dent. Res., 80:466-469, 2001.
Doench et al., Genes Dev., 17:438-42, 2003.
Dumoutier et al., J. Immunol., 164:1814-1819, 2000.
Eicher et al., Clin. Cancer Res., 2:1659-1664, 1996.
Ekmekcioglu et al., Int. J. Cancer, 94:54-59, 2001.
Ekmekciouglu et al., Melanoma Res., 9:261-272, 1999.
Elbashir et al., Nature, 411(6836):494-498, 2001.
Elroy-Stein et al., Proc. Natl. Acad. Sci. USA, 86:6126-6130, 1989.
Fausnaugh and Regnier, J Chromatog., 359:131-146, 1986.
Finn, Nat. Rev. Immunol., 3:630-641, 2003.
Fire et al., Nature, 391:806-811, 1998.
Flotte and Carter, Gene Ther., 2:357-62, 1995.
Flotte et al., Am. J. Respir. Cell Mol. Biol., 7:349-356, 1992.
Flotte et al., Proc. Natl. Acad. Sci. USA, 90:10613-10617, 1993.
Forster and Symons, Cell, 49:211-220, 1987.
Gallagher et al., Genes Immun., 1:442-450, 2000.
Gerlach et al., Nature (London), 328:802-805, 1987.
Ghosh-Choudhury et al., EMBO J., 6:1733-1739, 1987.
Gilbert, In: Adaptation of cells to serum free culture for production of adenovirus vectors and recombinant proteins, Williamsburg BioProcessing Conference, Nov. 18-21, 1996.
Glorioso et al., Mol. Biotechnol., 4:87-99, 1995.
Gomez-Foix et al., J. Biol. Chem., 267:25129-25134, 1992.
Graham and Prevec, Biotechnology, 20:363-390, 1992.
Graham and Prevec, In: Methods in Molecular Biology: Gene Transfer and Expression Protocol, Murray (Ed.), Humana Press, NJ, 7:109-128, 1991.
Graham et al., J. Gen. Virl., 36:59-74, 1977.
Griffiths, J. B., In “Animal Cell Biotechnology”, vol. 3, p 179-220, (Eds. Spier, R. E. and Griffiths, J. B.), Academic Press, London., 1986
Grishok et al., Cell, 106:23-34, 2001.
Grishok et al., Science, 287:2494-2497, 2000.
Grunhaus and Horwitz, Seminar in Virology, 3:237-252, 1992.
Guo et al., J. Dent. Res., 83:266-270, 2004.
Harlow and Lane, In: Antibodies. A laboratory Manual, Cold Spring Harbor Laboratories, Cold Spring Harbor, N.Y., 1988.
Hellstrom and Hellstrom, Expert Rev. Vaccines, 2:517-532, 2003.
Hermonat and Muzycska, Proc. Natl. Acad. Sci. USA, 81:6466-6470, 1984.
Hersdorffer et al., DNA Cell Biol., 9:713-723, 1990.
Herz and Gerard, Proc. Natl. Acad. Sci. USA, 90:2812-2816, 1993.
Hewish and Shukla, J. Virol. Methods, 7:223-228, 1983.
Holland and Holland, J Biol Chem, 255:2596-605, 1980.
Honess and Roizman, J. Virol., 14:8-19, 1974.
Honess and Roizman, J. Virol., 16:1308-130826. 1975.
Horwich, et al., J. Virol., 64:642-650, 1990.
Hutvagner and Zamore, Science, 297:2056-2060, 2002.
Hutvagner et al., Science, 293:834-838, 2001.
Huyghe et al., Human Gene Therapy, 6:1403-1416, 1996.
Jiang et al., Nature, 375:151-155, 1995.
Jiang et al., Proc. Natl. Acad. Sci. USA, 93:9160-9165, 1996.
Jones and Shenk, Cell, 13:181-188, 1978.
Joyce, Nature, 338:217-244, 1989.
Kaplitt et al., Nat. Genet., 8:148-154, 1994.
Karlsson et al., EMBO J., 5:2377-2385, 1986.
Ketting et al., Cell, 99:133-141, 1999.
Ketting et al., Genes Dev., 15:2654-2659, 2001.
Kim and Cook, Proc. Natl. Acad. Sci. USA, 84:8788-8792, 1987.
Knapp et al., Atherosclerosis, 152:217-227, 2000.
Knight and Bass, Science, 293:2269-2271, 2001.
Kornberg and Baker, DNA Replication, 2nd Ed., Freeman, San Francisco, 1992.
Kotenko et al., Proc. Natl. Acad. Sci. USA, 97:1695-1700, 2000.
Kotin et al., Proc. Natl. Acad. Sci. USA, 87:2211-2215, 1990.
LaFace et al., Virology, 162:483-486, 1988.
Laughlin et al., J. Virol., 60:515-524, 1986.
Le Gal La Salle et al., Science, 259:988-990, 1993.
Lebkowski et al., Mol. Cell. Biol., 8:3988-3996, 1988.
Levenson et al., Hum. Gene Ther., 9:1233-1236, 1998.
Levrero et al., Gene, 101:195-202, 1991.
Lin and Avery, Nature, 402:128-129, 1999.
Macejak and Sarnow, Nature, 353:90-94, 1991.
Mann et al., Cell, 33:153-159, 1983.
Markowitz et al., J. Virol., 62:1120-1124, 1988.
McCarty et al., J. Virol., 65:2936-2945, 1991.
McLaughlin et al., J. Virol., 62:1963-1973, 1988.
Michel and Westhof, J. Mol. Biol., 216:585-610, 1990.
Mizrahi, Dev Biol Stand., 55:219-230, 1983.
Montgomery et al., Proc. Natl. Acad. Sci. USA, 95:15502-15507, 1998.
Morris et al., “Serum-free production of adenoviral vectors for gene therapy,” Williamsburg BioProcessing Conference, Nov. 18-21, 1996.
Mourelatos et al., Genes Dev., 16:720-728, 2002.
Muzyczka, Curr. Topics Microbiol. Immunol., 158:97-129, 1992.
Nicolas and Rubenstein, In: Vectors. A survey of molecular cloning vectors and their uses, Rodriguez and Denhardt (Eds.), Stoneham: Butterworth, 494-513, 1988.
Ohi et al., Gene, 89:279-282, 1990.
Paskind et al., Virology, 67:242-248, 1975.
Pelletier and Sonenberg, Nature, 334(6180):320-325, 1988.
Perrin et al., Vaccine, 13:1244-1250, 1995.
Phillips et al., In: Large Scale Mammalian Cell Culture, (Feder, J. and Tolber (W. R., eds.), Academic Press, Orlando, Fla., U.S.A., 1985.
Post et al., Cell, 24:555-65, 1981.
Racher et al., Biotechnology Techniques, 9:169-174, 1995.
Ragot et al., Nature, 361:647-650, 1993.
Reinhart et al., Nature, 403:901-906, 2000.
Reinhold-Hurek and Shub, Nature, 357:173-176, 1992.
Renan, Radiother. Oncol., 19:197-218, 1990.
Rich et al., Hum. Gene Ther., 4:461-476, 1993.
Robbins and Ghivizzani, Pharmacol. Ther., 80:35-47, 1998.
Rosenfeld et al., Science, 252:431-434, 1991.
Rosenfeld, et al., Cell, 68:143-155, 1992.
Sambrook et al., In: Molecular cloning, Cold Spring Harbor Laboratory Press, Cold Spring Harbor, N.Y., 2001.
Samulski et al., EMBO J., 10:3941-3950, 1991.
Samulski et al., J. Virol., 63:3822-3828, 1989.
Sarver et al., Science, 247:1222-1225, 1990.
Saxena et al., J. Biol. Chem., 278:44312-44319, 2003.
Scanlon et al., Proc. Natl. Acad. Sci. USA, 88:10591-10595, 1991.
Schaefer et al., Cell Signal, 12:143-151, 2000.
Scheit, In: Synthesis and Biological Function, Wiley-Interscience, NY, 171-172, 1980.
Sharp and Zamore, Science, 287:2431-2433, 2000.
Sharp, Genes Dev., 13:139-141, 1999.
Shelling and Smith, Gene Therapy, 1:165-169, 1994.
Smith and Moss, Gene, 25(1):21-28, 1983.
Soo et al., J. Cell. Biochem., 74:1-10, 1999.
Stratford-Perricaudet and Perricaudet, In: Human Gene Transfer, Eds, Cohen-Haguenauer and Boiron, John Libbey Eurotext, France, 51-61, 1991.
Stratford-Perricaudet et al., Hum. Gene. Ther., 1:241-256, 1990.
Tabara et al., Cell, 99:123-132, 1999.
Tang et al., Blood, 104:2704-2713, 2004.
Tarnawski, Dig. Dis. Sci., 50 Suppl 1:S24-33, 2005.
Temin, In: Gene Transfer, Kucherlapati (Ed.), NY, Plenum Press, 149-188, 1986.
Top et al., J. Infect. Dis., 124:155-160, 1971.
Tratschin et al., Mol. Cell. Biol., 4:2072-2081, 1984.
Tratschin et al., Mol. Cell. Biol., 5:3258-3260, 1985.
van Wezel, Nature, 216:64-65, 1967.
Vogelstein et al., Nature, 408:307-310, 2000.
Walsh et al., J. Clin. Invest, 94:1440-1448, 1994.
Wei et al., Gene Therapy, 1:261-268, 1994.
Xu et al., Curr. Biol., 13:790-795, 2003.
Yang et al., J. Virol., 68:4847-4856, 1994.
Yoder et al., Blood, 82 (Supp.): 1:347A, 1994.
Zeng et al., Cancer Res., 62:3630-3635, 2002.
Zhang et al., Endocrinology, 141:4698-4710, 2000.
Zhou et al., Exp. Hematol, 21:928-933, 1993.
Zhou et al., J. Exp. Med., 179:1867-1875, 1994.

What is claimed is:

1. A method for evaluating a virus sample previously purified by means other than size exclusion chromatography, comprising:
(a) subjecting a virus sample to high performance size exclusion chromatography, thereby producing an eluate;
(b) subjecting the eluate to spectroscopy; and
(c) evaluating the eluate by one or more of the following:
(i) quantifying the virus in the sample by determining an area of peaks representing virus-containing peak or peaks;
(ii) qualifying the sample by assessing symmetry of the virus-containing peak or peaks; or
(iii) assessing purity of the sample by dividing virus-containing peak or peaks area by total peak area.

2. The method of claim 1, wherein a chromatographic medium used in the high performance size exclusion chromatography has a pore size of about 5 to 100 nm.

3. The method of claim 2, wherein the size exclusion chromatographic medium has a pre size of about 29 nm.

4. The method of claim 1, wherein the spectroscopy is wavelength absorbance.

5. The method of claim 4, wherein the wavelength is about 215 nm to about 280 nm.

6. The method of claim 5, wherein the wavelength is about 215 nm.

7. The method of claim 1, wherein the spectroscopy is refractive index.

8. The method of claim 1, wherein the spectroscopy is fluorescence emission.

9. The method of claim 1, wherein the spectroscopy is light scattering.

10. The method of claim 1, wherein the virus sample was previously purified by ultracentrifugation, ion exchange chromatography, immobilized metal affinity chromatography, sulfated affinity chromatography, immunoaffinity chromatography, heparin affinity chromatography, hydrophobic interaction chromatography, hydroxyapatite chromatography or combinations thereof.

11. The method of claim 10, wherein the virus sample was previously purified by ion exchange chromatography.

12. The method of claim 11, wherein the ion exchange chromatography is anion exchange chromatography.

13. The method of claim 12, wherein a chromatographic medium used in the anion exchange chromatography is Amersham Biosciences Source 15Q.

14. The method of claim 1, wherein the previously purified virus sample was stored prior for a time period between one day and two years prior to analyzing the virus sample via high performance size exclusion chromatography.

15. The method of claim 1, wherein the virus sample is an adenovirus sample, an adeno-associated virus sample, a retrovirus sample, a herpesvirus sample, a pox virus sample, a picornavirus sample or an alphavirus sample.

16. The method of claim 15, wherein the virus sample is an adenovirus sample.
